(19) United States
(12) Patent Application Publication
(10) Pub. No.: US 2026/0144916 A1
(43) Pub. Date: May 28, 2026

(54) METHOD AND SYSTEM FOR DESIGNING AND PLANNING FIXATION OF AN IMPLANTABLE SCAFFOLD FOR BONE REGENERATION

(71) Applicants: Alexander Natanzon, Upper Saddle River, NJ (US); Arthur Greyf, New York, NY (US)

(72) Inventors: Alexander Natanzon, Upper Saddle River, NJ (US); Arthur Greyf, New York, NY (US)

(21) Appl. No.: 18/956,051

(22) Filed: Nov. 22, 2024

(51) Int. Cl. A61L 27/50

(52) U.S. Cl. CPC A61L 27/50

(57) ABSTRACT

A scaffold designing engine (SDE) and a method for designing an outer shape of an implantable scaffold (IS), are provided. The SDE identifies a digital representation of a defect contour separating a healthy bone surface from an outer surface of a bone defect using complementary, cross-sectional image visualizations and/or a three-dimensional image visualization of a data volume, and renders a digital representation of the outer surface of the bone defect as a membrane. The SDE identifies a spatial location of a first base plane and a base facet of the IS. The SDE renders a tubular surface connecting the outer contour of the base facet and the defect contour as a wall surface and a digital representation of a two-dimensional outer surface of the IS by a contiguous joinder of two-dimensional segments of the base facet, the membrane, and a wall surface.

### BACKGROUND

Bone regeneration is required to preclude bone from deteriorating in the absence of tooth roots that secure a portion of a person's jaw. After a person loses a tooth, the bone that surrounded the tooth typically deteriorates, decreasing in width, height, and density. The longer the tooth has been missing, the more the bone that used to surround the tooth resorbs. A dental bone graft is typically needed when persons have bone loss in their jaws. Dental bone grafting is an oral surgery procedure that adds density and volume to a jawbone in areas where bone loss has occurred. Dental bone grafting is used to build up or regenerate new bone in the area of the jaw that used to hold teeth. Dental bone grafting is typically performed, for example, prior to a tooth extraction, prior to replacement of a missing tooth with a dental implant, prior to rebuilding of the jaw for dentures, or in cases of having areas of bone loss due to periodontal disease that affect the health of nearby gums and teeth. The dental bone graft allows bone tissue to grow and regenerate thereon. In a typical dental bone grafting procedure, a medical practitioner, for example, a dentist, creates a small incision in a patient's gums, moves back gum tissue slightly to expose the bone beneath the gums, adds bone grafting material to repair a bone defect, covers the dental bone graft with a membrane for additional protection, repositions the gum tissue, and closes the incision with stitches. The bone grafting material comprises processed bone, for example, bone from the hip, tibia, or back of the jaw, around which new bone cells are deposited. The bone grafting material is eventually absorbed by the body and replaced by new bone. Bone regeneration uses the bone grafting material to grow new bone in weak areas of the jawbone.

A bone defect may be in the form of a void surrounded 360 degrees by walls, or a void surrounded less than 360 degrees by walls, or a void having missing or no walls. Dental bone grafting is typically performed to grow bone in the void surrounded 360 degrees by walls. When bone regeneration is required, dental bone grafting is performed by applying the bone grafting material or paste directly into the void surrounded 360 degrees by walls with bone material. When the void is surrounded less than 360 degrees by walls or has a missing wall, the success rate for bone regeneration substantially decreases, as it is difficult to apply the bone grafting material in the inadequate void. Alternative to dental bone grafting, scaffolding is performed, when bone regeneration is planned, not for a confined void, but rather to increase the thickness of the bone or to amend a missing part of the bone. In the case of scaffolding, a dedicated spatial structure similar to a high-rise building metal skeleton is used to contain and confine the bone grafting material in its inter-cellular volumes. This dedicated spatial structure should be shaped according to the shape and volume of the desired bone augmentation and then attached to the bone itself.

Computer-aided design (CAD) and computer-aided manufacturing (CAM), that are widely utilized for machinery and construction design, have emerged as a part of dentistry to improve the design and construction of dental restorations, for example, crowns, inlays, onlays, veneers, bridges, dentures, implant abutments, dental prostheses, and implant-supported restorations from high-strength ceramic, resin, or metal. Most conventional CAD/CAM applications that are capable of designing implants, were configured originally for wide profile engineering design. These CAD/CAM applications typically employ standard engineering concepts and methodologies for designing which presume that each complex component is constructed from standard engineering elements. Each engineering element is constructed with its surface rendered as a specialized part using one of the standard engineering formats, for example, a stereolithography (STL) file format. Once this elementary part is rendered, the elementary part is automatically added to a parts database or a list, for example, a bill of materials (BOM). The complex part is then constructed as a combination of the elementary parts selected from the BOM. Although this construction approach has proven effective for engineering purposes when each engineering element is designed once, without interactive changes or adjustments, this approach is substantially ineffective for designing implantable prosthetic elements which require multiple iterative adjustments, after the complex part is fully rendered. This results in a tedious iterative process, where a medical practitioner is forced to roll back a project several steps to redesign each elementary part, then rebuild the parts database, and only then assemble the final complex part from the re-engineered elementary parts.

Most commercially available CAD software packages utilize concepts and methods that typical medical practitioners are not trained on or even aware of. Moreover, most commercially available CAD software packages offer a universal solution suitable for a wide variety of engineering tasks and therefore do not offer any seamless workflow for designing implantable scaffolds based on computer tomography (CT) or cone beam computed tomography (CBCT) images delivered in a Digital Imaging and Communications in Medicine (DICOM) format. Although most of these CAD software packages are capable of producing implant design, such design requires multiple and cumbersome steps that are usually too complicated for implementation by a medical practitioner without special engineering knowledge and training. Lack of special engineering knowledge and training in CAD precludes a medical practitioner from generating a “fully inhouse” design of an implantable scaffold and from producing the implantable scaffold through seamless and time-concise steps. There is a need for a targeted software solution for designing an outer shape of an implantable scaffold, that implements specific design steps in one seamless workflow tuned to the specifics of the geometrical design of bone regeneration implants, which allows for printing the implantable scaffold on an extrusion three-dimensional (3D) printer, and that can be conveniently and comfortably used by general medical practitioners, for example, physicians, dental, orthopedic or general surgeons, practicing dentists, etc., who do not possess specific skills and knowledge typical to engineers trained for CAD software.

Hence, there is a long-felt need for a method and a system for designing an implantable scaffold for bone regeneration in a seamless workflow tuned to the specifics of the geometrical design of bone regeneration implants, which allows for printing the implantable scaffold on an extrusion 3D printer, where the implantable scaffold is configured to fit the shape and volume of the bone defect or the void to create walls therefor. Furthermore, there is a long-felt need for a method and a system for planning fixation of the implantable scaffold, while preventing protrusion of a fixation member, for example, a fixation screw, beyond a necessary anchoring volume into a dangerous proximity to vulnerable anatomical structures identified in the vicinity of the implantable scaffold.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of the invention is better understood when read in conjunction with the appended drawings. For illustrating the embodiments herein, exemplary constructions of the embodiments are shown in the drawings. However, the embodiments herein are not limited to the specific methods, components, and structures disclosed herein. The description of a method step, or a component, or a structure referenced by a numeral in a drawing is applicable to the description of that method step, or that component, or that structure shown by that same numeral in any subsequent drawing herein.

FIG. 1A illustrates a flowchart of an embodiment of a method for designing an implantable scaffold.

FIG. 1B illustrates a flowchart of an embodiment of a method for planning fixation of an implantable scaffold.

FIGS. 2A-2T illustrate screenshots of graphical user interfaces rendered by a scaffold designing engine for designing and planning fixation of an implantable scaffold.

FIGS. 3A-3E illustrate complex geometries of individual linear struts and bars encapsulated by an implantable scaffold.

FIG. 4 illustrates an architectural block diagram of an exemplary implementation of a system comprising a scaffold designing engine for designing and planning fixation of an implantable scaffold.

### DETAILED DESCRIPTION OF THE INVENTION

Various aspects of the disclosure herein are embodied as a method, a system, or a non-transitory, computer-readable storage medium having one or more computer-readable program codes stored thereon. Accordingly, various embodiments of the disclosure herein take the form of an entirely hardware embodiment, an entirely software embodiment comprising, for example, microcode, firmware, software, etc., or an embodiment combining software and hardware aspects that are referred to herein as a “system”, a “module”, an “engine”, or a “unit”. The terms “first” and “second” are used herein for descriptive purposes only and are not to be construed to indicate or imply relative importance.

In one or more embodiments, related systems comprise circuitry and/or programming for executing the methods disclosed herein. The circuitry and/or programming comprise one or any combination of hardware, software, and/or firmware configured to execute the methods disclosed herein depending upon the design choices of a system designer. In an embodiment, various structural elements are employed depending on the design choices of the system designer.

The method and the system disclosed herein address the long-felt need for designing an implantable scaffold for bone regeneration in a seamless workflow tuned to specifics of a geometrical design of bone regeneration implants, which allows for printing the implantable scaffold on an extrusion three-dimensional (3D) printer, where the implantable scaffold is configured to fit the shape and volume of a bone defect or a void to create walls therefor. The method and the system disclosed herein also address the long-felt need for planning fixation of the implantable scaffold, while preventing protrusion of a fixation member, for example, a fixation screw, beyond a necessary anchoring volume into a dangerous proximity to vulnerable anatomical structures identified in the vicinity of the implantable scaffold.

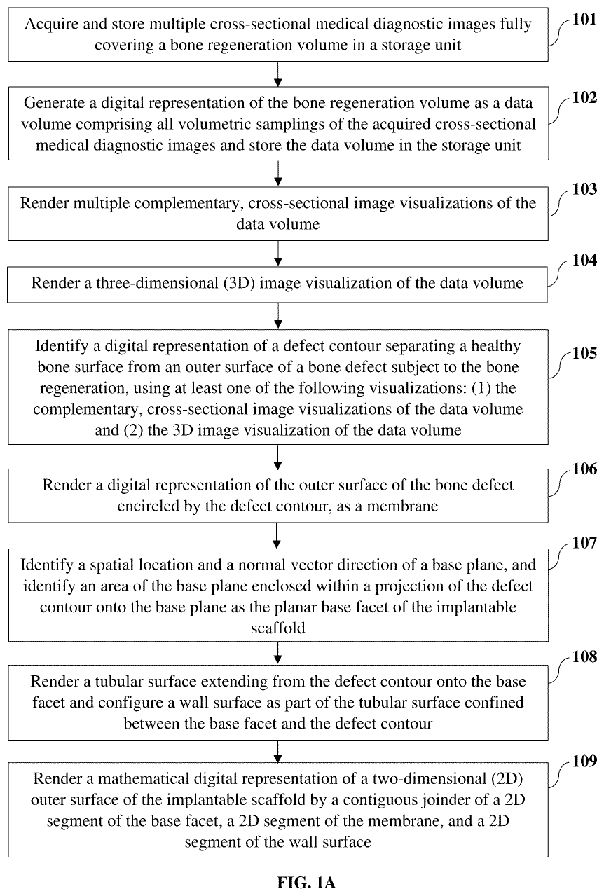

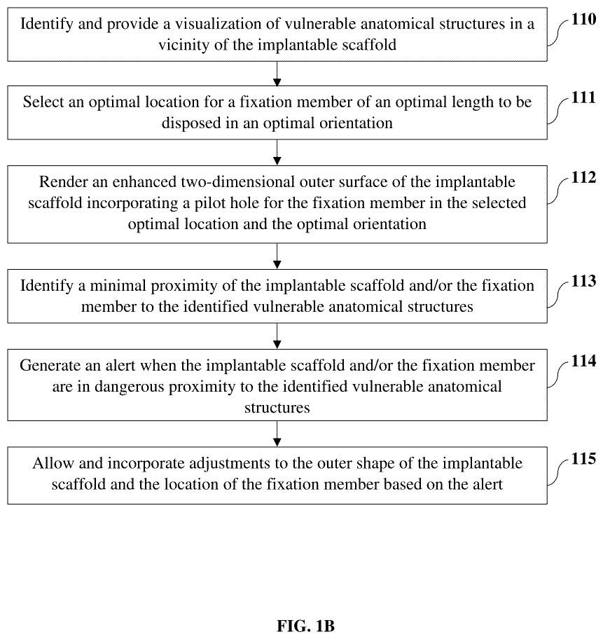

FIG. 1A illustrates a flowchart of an embodiment of a method for designing an implantable scaffold. As used herein, “implantable scaffold” refers to a bone regeneration implant configured to fit the shape and volume of a bone defect or a void. The implantable scaffold is a substantially porous structure similar to a honeycomb made, for example, of a resorbable plastic. The implantable scaffold is implanted into a bone regeneration area and optionally holds a bone grafting material in its pores. The outer shape of the implantable scaffold is intimately tied to the bone defect. The implantable scaffold assists in bone regeneration by creating one or more walls for the bone defect. In an exemplary embodiment, the implantable scaffold is a computer-aided design (CAD)-designed, three-dimensionally-printed, honeycomb-shaped implant optionally filled with a bone grafting material selected by a medical practitioner. In this embodiment, the implantable scaffold is a dedicated spatial structure used to contain and confine the bone grafting material in its inter-cellular volumes. Instead of applying the bone grafting material into the bone defect directly, the medical practitioner smears the bone grafting material into cells of the implantable scaffold. In another exemplary embodiment, the implantable scaffold is a CAD-designed, three-dimensionally-printed, honeycomb-shaped implant without any filling of a bone grafting material. In this embodiment, the implantable scaffold is a dedicated spatial structure used to serve as a placeholder and bone growth accelerator by itself without any filling. The implantable scaffold is configured to fit a patient's anatomy with a preplanned fixation screw hole. The resorbable plastic of the implantable scaffold is washed away from the patient's body, for example, in about three to six months. The implantable scaffold assists in creating walls for a bone defect that is, for example, surrounded less than 360 degrees by walls, or that has a missing wall. The implantable scaffold assists in increasing the thickness of the bone and amending a missing part of the bone. The implantable scaffold serves as a placeholder for the newly regenerated bone and prevents soft tissues invagination into that preserved and properly shaped volume. The implantable scaffold is shaped according to the shape and volume of the desired bone augmentation and then attached to the bone itself.

The method disclosed herein provides a targeted software solution for designing an outer shape of an implantable scaffold, that implements specific design steps in one seamless workflow tuned to the specifics of the geometrical design of a bone regeneration implant, and that can be conveniently and comfortably used by general medical practitioners, for example, physicians, dental, orthopedic or general surgeons, practicing dentists, etc., who do not possess specific skills and knowledge typical to engineers trained for CAD software. In an exemplary embodiment, the implantable scaffold is three-dimensionally-printable on an extrusion three-dimensional (3D) printer. Printing may be performed at the patient's bedside, for example, in less than 30 minutes. In another exemplary embodiment, the implantable scaffold is a non-3D-printable scaffold. In an exemplary embodiment, the method disclosed herein allows a medical practitioner to generate a fully inhouse design of an implantable scaffold and produce the implant scaffold through seamless and time-concise steps. In another exemplary embodiment, the method disclosed herein allows a medical practitioner to order a printable, implantable scaffold from a third party, that produces the implantable scaffold through seamless and time-concise steps and delivers the implantable scaffold to the medical practitioner.

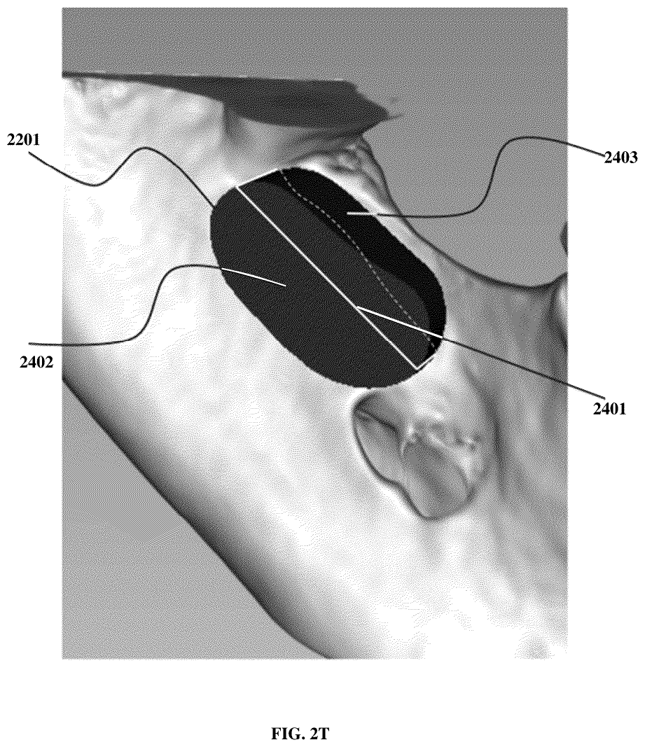

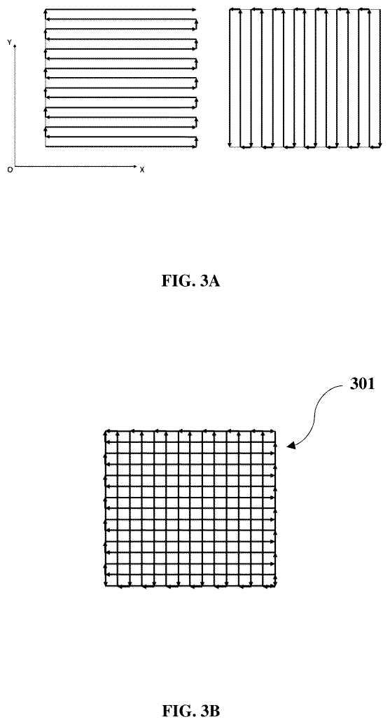

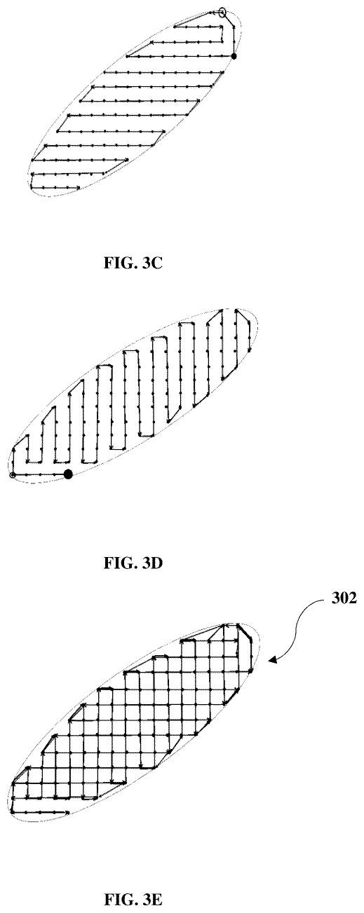

In an exemplary embodiment, the method disclosed herein employs one or more of mathematical algorithms and/or artificial intelligence (AI) algorithms comprising deep learning methods for implementing one or more of the steps for designing the outer shape of the implantable scaffold disclosed below. In another exemplary embodiment, the method disclosed herein employs user instructions for implementing one or more of the steps for designing the outer shape of the implantable scaffold disclosed below. The method disclosed herein employs a scaffold designing engine configured to implement computer program instructions executable by at least one processor for designing an outer shape of an implantable scaffold for bone regeneration. The scaffold designing engine designs the outer shape of the implantable scaffold by designing an outer envelope, also referred to as a “shell”, configured to encapsulate a complex geometry of individual linear struts and bars separated by air-filled gaps as illustrated in FIGS. 3A-3E, which provides a porous structure required for optimal bone ingrowth and for optional initial placement of bone grafting material, for example, bone grafting pastes, and for further ingrowth of new bone, and a vascular and nerve network. The complex geometry of individual linear struts is designed to provide the following: (a) the linear struts are tightly inscribed into the shell; and (b) interconnections of the linear struts, while providing the needed structural rigidity, still represent a highly porous volumetric structure with high permeability of its outer surface and inner volume.

The scaffold designing engine designs the outer shape of the implantable scaffold such that: (1) the outer shape of the implantable scaffold intimately matches the shape of a bone defect or a void that the implantable scaffold intends to fill; and (2) the geometrical design of the outer shape allows for straightforward printing on an extrusion three-dimensional (3D) printer, wherein: (a) a flat surface, for example, a printing table, is used for starting a 3D buildup, with the first layer being extruded right onto the printing table; and (b) each subsequent layer is completely contained within an outer circumference of the previous layer so that newly extruded hot polymer bead has a solid rest over the already cooled struts and bars of the previous layer. The above requirement (2) defines that the 3D printed part must have at least one flat base facet; vertical or converging walls rising perpendicular to that base facet or inward towards its inner area; and a 3D roof-top or dome of any type resting over the vertical walls. For intimate fitting of the implantable scaffold to a surface of a bone defect, the 3D roof-top or dome is the only part of the printable shape that matches the above requirements (1) and (2).

Production of the implantable scaffold is performed through the following seamless and time-concise steps:
(1) Acquiring a complete set of volumetric cross-sectional computer tomography (CT) or cone beam computed tomography (CBCT) images of a desired anatomical area, which are acquired, stored and accessible in a Digital Imaging and Communications in Medicine (DICOM) format; and porting the acquired DICOM-formatted images into a computer system;
(2) Engineering a desired outer shape of the implantable scaffold through a dedicated seamless workflow feasible for an average trained medical practitioner;
(3) Porting the engineered outer shape into another piece of fully automated non-interactive software configured to:
   (a) create an internal struts geometry inscribed into the engineered outer shape and printable on a 3D printer; and
   (b) transform the internal struts geometry into a step-by-step printing script readable by the 3D printer in a customary industry-wide encoding standard, for example, G-code, and port the printing script into the 3D printer, for example, an in-house 3D printer or a third-party 3D printer; and
(4) Printing the implantable scaffold featuring the desired outer shape and internal struts structure as encoded in the imported G-code printing script file, on the 3D printer suited for a use case. In an exemplary embodiment, the 3D printer produces a sterile, ready-to-implant, bone regeneration implant or implantable scaffold without any additional sterilization steps.

The scaffold designing engine disclosed herein is configured to execute step (2) of the above-disclosed time-concise steps for producing the implantable scaffold. That is, the scaffold designing engine is configured to engineer the desired outer shape of the implantable scaffold through a dedicated seamless workflow feasible for an average trained medical practitioner as disclosed below. In an exemplary embodiment, the scaffold designing engine is configured to execute one or more of mathematical algorithms and AI algorithms comprising deep learning methods for implementing one or more steps of the method for designing the outer shape of the implantable scaffold disclosed below. As shown in FIG. 1, in the method disclosed herein, the scaffold designing engine acquires and stores 101 multiple cross-sectional medical diagnostic images, for example, computer tomography (CT) images, cone beam computed tomography (CBCT) images, etc., fully covering a bone regeneration volume in a storage unit. The scaffold designing engine then generates 102 a digital representation of the bone regeneration volume as a data volume comprising volumetric samplings of the cross-sectional medical diagnostic images and stores the data volume in the storage unit. Each of the acquired cross-sectional medical diagnostic images represents a multiplicity of sampling values sampled over a uniform planar rectangular grid, while all images of the stack provide the sampling over parallel equidistant planar grids covering a cubic-shaped volume. The data volume is a multi-dimensional data structure configured to store data in more than two dimensions. More particularly, the data volume is a spatial contiguous volume covered with sampled CT or CBCT volumetric data in a uniform manner allowing for a substantially accurate interpolation of the sampled CT or CBCT volumetric data between sampling points in any direction within that volume and thus building various representations of the spatial volumetric data comprising, for example, planar or curved cross-sections. In an example, when the scaffold designing engine acquires the original cross-sectional medical diagnostic images as a set of parallel equidistant planes sampled on a two-dimensional (2D) image matrix with equidistant rows and columns, the data volume is a concatenation of the original data 2D image matrices. If the original cross-sectional medical diagnostic images are either non-parallel, or sampled on a non-equidistant image matrix, or if the distance between the planes is different from the distance between rows and columns in each image matrix, the data volume is a resampling of the original data on the 3D Cartesian equidistant lattice.

The scaffold designing engine then renders 103 multiple complementary, cross-sectional image visualizations of the data volume and renders 104 a 3D image visualization of the same data volume, for example, using standard techniques of resampling, creating iso-value surfaces, ray tracing, etc., used in the medical imaging industry and known to those skilled in the art. As used herein, “3D image visualization” refers to a visual presentation of a general topology and a detailed structure of local features on a surface of a bone as registered on CT or CBCT cross-sectional data facilitating a user to not only receive these details but also to pinpoint a 3D location of any selected landmark on that surface. The scaffold designing engine creates the complementary, cross-sectional image visualizations of the data volume and the 3D image visualization of the data volume using standard commercially available software development kits (SDKs) or shareware, for example, the 3D Slicer image computing platform. In one method, a user selects a typical CT number that segregates between bones and soft tissues, where soft tissues have lesser values and bones have higher values. The scaffold designing engine then performs segregation between two sub-volumes within the data volume, where one sub-volume features soft tissue values and the other sub-volume features bone values. The scaffold designing engine then denotes the surface separating the two sub-volumes as a bone surface and generates a visualization in a 3D perspective using standard methods of visualizations known to artists, architects, engineers, etc., and those skilled in the art.

Yet another method of visualization is called volumetric visualization. According to the method of volumetric visualization, the scaffold designing engine attributes each voxel within the data volume with certain imaginary optical properties comprising, for example, intrinsic luminosity, optical opacity, light scattering, and light reflectivity. The scaffold designing engine sets these parameters as a function of a local CT value, for example, through a ramp function so that all voxels with values less than a foot value are absolutely transparent to light; all voxels with values above the top value of the ramp function are totally opaque; while those voxels with values in the middle have an intermediate opacity and luminosity depending on their position on the ramp. All rays emitted by a distant source are then traced as they travel through the data volume towards a projection screen and the light intensity of each ray is updated as each ray passes through each next voxel. The light intensity of each ray is attenuated according to the local opacity and scatter and, respectively enhanced if the voxel is attributed with some local luminosity. This process appears to simulate the passage of sunlight through a thunder cloud which has no internal luminosity, however, local opacity of which is proportional to the respective local water and ice content within that area of the cloud.

In an exemplary embodiment, a method of identifying or marking specific seed points or landmarks, herein referred to as contour points seeding or landmark seeding, within the 3D image visualization, is performed either manually or through automated algorithms. These seed points or landmarks serve as reference points from which the visualization or analysis begins. Landmark seeding on the 3D image visualization typically starts with a mouse click on a screen point featuring a desired landmark. If the 3D visualization is performed as a visualization of the spatial surface, then the built-in scaffold designing engine identifies the respective point of the visualized bone surface that falls on the point of the mouse click. Alternatively, when a user intends to identify a spatial location on a volumetric cloud-like rendering of the bone surface, the following procedure is simulated. In an exemplary embodiment, the scaffold designing engine executes a “ray back projection” algorithm. By executing the ray back projection algorithm, the scaffold designing engine runs an imaginary ray from a selected point of the projection screen towards a light source and calculates a light attenuation of an imaginary test light moving along this line. At first, light attenuation is negligible, but as the test point starts to dip into the volume with considerable opacity, total attenuation starts growing drastically. As it happens, the scaffold designing engine stops this ray back projection and sets the last test location as the location of an optical border.

The complementary cross-sectional image visualizations comprise planar cross-sectional sampling of the data volume. The three-dimensional image visualization of the data volume comprises one of: visualizations of three-dimensional (3D) data iso-surfaces built over data of the data volume, visualizations of two-dimensional (2D) projections of ray tracing models built over the data of the data volume, and any combination thereof. The scaffold designing engine then identifies 105 a digital representation of a defect contour separating a healthy bone surface from an outer surface of a bone defect subject to the bone regeneration, using at least one of the rendered complementary, cross-sectional image visualizations of the data volume and the 3D image visualization of the data volume. As used herein, “defect contour” refers to a spatially non-planar contour line separating an area subjected to bone regeneration from a healthy area. In an example, to identify a digital representation of a defect contour, a user first seeds multiple 3D points on the 3D image visualization of the data volume, where each point belongs to the defect contour according to the user's assessment. The scaffold designing engine then applies standard 3D interpolation methods known in the art for creating a 3D smooth curve that passes through the seed points as the digital representation of the defect contour. The digital representation of the defect contour comprises multiple sampling points or seed points belonging to the defect contour, that are connected with smooth 3D analytically interpolating curves. The scaffold designing engine constructs the smooth 3D analytically interpolating curves, for example, using parametric curves such as Bézier curves used in computer graphics, where a set of discrete control points defines a smooth, continuous curve by means of a formula. In an exemplary embodiment, the scaffold designing engine facilitates an interactive identification of isolated seed points of the defect contour with a subsequent smooth interpolation between these isolated seed points by any customary means used in computer graphics, for example, using Bézier curves.

Furthermore, in the method disclosed herein, the scaffold designing engine renders 106 a mathematical digital representation of the outer surface of the bone defect encircled by the defect contour, as a membrane. As used herein, the term “membrane” refers to a bone surface enclosed by the defect contour. The scaffold designing engine creates the membrane by identifying a two-dimensional (2D) area of the bone surface circumferenced by the defect contour. The membrane is the upper surface of the implantable scaffold that is attached directly to the exposed healthy bone surface of the bone defect or the bone regeneration area. This upper surface should not be tight to the bone surface along its whole area, but rather should be tight only along its perimeter that should be reasonably air-tight to the defect contour to allow for unique positioning of the implantable scaffold into the bone regeneration area. It is beneficial to have some unfilled space between the upper surface of the implantable scaffold and the bone surface, as this unfilled space can be filled with the bone grafting material or paste confined by the body of the implantable scaffold, without carrying any foreign bodies that obstruct contiguous bone growth.

In an exemplary embodiment, the scaffold designing engine defines the membrane through a three-dimensional (3D) interpolation of the defect contour into an interior of the defect contour using standard methods of modern computer graphics. For example, one such method of interpolation simulates a spatial equilibrium of an elastic film tightly stretched over the rigid shape of the defect contour. In another exemplary embodiment, the 3D interpolation of the defect contour into the interior of the defect contour comprises employing interactively seeded anchor points in the membrane. In an exemplary embodiment, the scaffold designing engine performs the 3D interpolation of the defect contour by a slight modification of the simulation of the elastic film equilibrium shape. The modification of the simulation of the elastic film equilibrium shape comprises stretching the elastic film equilibrium shape over the defect contour and nailing the elastic film equilibrium shape to the interactively seeded and adjusted anchor points. In an exemplary embodiment, the membrane is configured to be adjacent to a bone surface only along and in the vicinity of the defect contour, while being considerably separated from the healthy bone surface within an inner area of the membrane. If the user is not satisfied with the position of the membrane to the bone surface, the scaffold designing engine allows the user to add anchor points on the membrane and seed the anchor points to tie the membrane firmly against the bone surface. Furthermore, the scaffold designing engine allows the user to partially separate the membrane from the bone surface to create a void for applying bone grafting material therein between the bone and the implantable scaffold. The scaffold designing engine allows the user to tighten only the edges of the membrane to the bone surface while leaving a central void which detaches the membrane from the bone surface. In another exemplary embodiment, the scaffold designing engine performs the 3D interpolation of the defect contour into the interior of the defect contour using one or more known mathematical algorithms and/or a mathematical function built over local data of the data volume.

The scaffold designing engine then identifies 107 a spatial location and a normal vector direction of a first base plane configured to hold a first printed layer of the implantable scaffold. As used herein, “normal vector” refers to a unitary vector whose direction is perpendicular to any straight line belonging to the first base plane. The scaffold designing engine then renders a projection of the defect contour onto the first base plane. The area of the first base plane enclosed within that projection of the defect contour becomes the first printed layer of the implantable scaffold and is herein referred to as a “base facet”. The scaffold designing engine identifies 107 the area of the first base plane enclosed within the projection of the defect contour onto the first base plane as the base facet of the implantable scaffold. As used herein, “base facet” refers to the flat base of the implantable scaffold, typically, the first layer of a 3D buildup that is extruded over a flat building plate of an extrusion 3D printer. All subsequent layers are extruded over this flat base. Also, as used herein, “first base plane” refers to a plane in space that holds the base facet when the implantable scaffold is tightly attached to the bone defect. The contour of the base facet which is the projection of the defect contour on the first base plane is herein referred to as “base contour”.

The scaffold designing engine renders 108 a first tubular surface extending from the defect contour onto the base facet. In an exemplary embodiment, the first tubular surface is a classical mathematical cylinder defined by the defect contour as a generatrix and by an extrusion vector that is substantially parallel to the normal vector to the first base plane. A border of the base facet is defined as a projection of the defect contour onto the first base plane along the extrusion vector. The scaffold designing engine then configures 108 a wall surface as part of the first tubular surface confined between the base facet and the defect contour to air-tightly embrace the base facet at a first cross-section of the wall surface and air-tightly embrace the defect contour at a second cross-section of the wall surface. As used herein, “wall surface” refers to a gradually growing side surface comprised from edges of each subsequent layer as each layer is extruded over the previous layers. As each extruded bead of a polymer should entirely rest over the previously extruded and already cooled layers, the extruded bead can protrude over the previous layer only a bit at its very end. The side walls of this build-up must, therefore, be vertical or converging to the top, or only slightly diverging with a reasonably minimal layer over layer hangover.

The scaffold designing engine then renders 109 a mathematical digital representation of a two-dimensional (2D) outer surface of the implantable scaffold by a contiguous joinder of 2D segments comprising a 2D segment of the base facet, a 2D segment of the membrane, and a 2D segment of the wall surface. The rendering of the mathematical digital representation of the 2D outer surface of the implantable scaffold constitutes the design of the outer shape of the implantable scaffold.

In an exemplary embodiment, user interactions are used for implementing one or more steps of the method for designing the outer shape of the implantable scaffold. The user interactions are performed over 2D and 3D computer visualizations rendered in the steps 102 through 109 of the method disclosed herein. For example, the user interactions are performed over a 3D image visualization of the bone regeneration volume, and three complementary, cross-sectional image visualizations. In another example, the user interactions are performed over the digital representation of the outer surface of the bone defect encircled by the defect contour, the first tubular surface, and the digital representation of the 2D outer surface of the implantable scaffold. In an exemplary embodiment, the scaffold designing engine employs three complementary, cross-sectional image visualizations along three mutually perpendicular cross-sectional planes, herein referred to as complementary planar views. As used herein, “complementary planar views” refers to cross-sectional image visualizations along three mutually perpendicular cross-sectional planes simultaneously visualized on three panels side by side on a graphical user interface rendered by the scaffold designing engine. Also, as used herein, “cross-sectional image visualization” refers to a stack of 2D images build using 2D samplings of an intersection of the data volume with multiple parallel planar surfaces cutting through the data volume. The scaffold designing engine renders each 2D image within the stack over a 2D pixelated matrix, where the value of a pixel is interpolated between neighboring sampling points of the original data volume. The scaffold designing engine then converts the resampled value in each pixel into a gray level using a ramp function where the actual sampling values corresponding to the bottom and to the top of the ramp, that is, the low/high window level, is interactively adjusted by a user for better perception of the actual dynamic range of the data sampled in the 2D pixelated matrix. In an exemplary embodiment, the scaffold designing engine interactively adjusts orientation of the three mutually perpendicular cross-sectional planes.

In another exemplary embodiment, the scaffold designing engine employs visualizations of the 3D data iso-surfaces within the data volume. In another exemplary embodiment, the scaffold designing engine employs visualizations of the iso-surfaces of a combination of the data within the data volume and its spatial derivatives to enhance an image. In another exemplary embodiment, the scaffold designing engine employs visualizations of the iso-surfaces of even more complex mathematical functions over the data volume than just the combination of the data within the data volume and its spatial derivatives to enhance the image. In another exemplary embodiment, the scaffold designing engine employs a visualization of a digital simulation of optical rays penetrating through an optically nonuniform volume, also referred to as an optical nonuniform cloud. The local optical characteristics are mathematically related to local values within the data volume.

FIG. 1B illustrates a flowchart of an embodiment of a method for planning fixation of an implantable scaffold. In the method disclosed herein, the scaffold designing engine identifies and provides 110 a visualization of vulnerable anatomical structures using one or more user interactions, artificial intelligence (AI) algorithms comprising deep learning algorithms, or any combination thereof. Upon surgical implantation of the scaffold, the area subjected to surgical intervention is usually wider than the immediate volume of the implantable scaffold. Usually, all areas of possible surgical intervention are carefully selected in such a way that disturbed tissues are fully regenerated upon post-surgery healing. However, certain tissues, for example, nerves, ligaments, etc., do not regenerate if disturbed, or cut, or otherwise damaged. Thus, pre-surgery planning involves the step of identification of such areas and avoidance of even a remote possibility of accidental surgical intervention in these areas or even within their close proximity. In an exemplary embodiment, a user identifies a surface of a mandible nerve as one of the vulnerable anatomical structures as follows. The user first seeds points along an axis of the mandible nerve, herein referred to as “spatial nerve axis”, for example, using three complementary, cross-sectional image visualizations. The scaffold designing engine then performs a smooth 3D interpolation between the seeded points, for example, using the Bézier interpolation, similar to the identification of the defect contour. The user then selects and adjusts a diameter of the mandible nerve via the graphical user interface rendered by the scaffold designing engine. The scaffold designing engine then creates a first tubular surface of a specified diameter around the defined spatial nerve axis. In an exemplary embodiment, the scaffold designing engine identifies a minimal proximity of the implantable scaffold to the vulnerable anatomical structures, and generates an alert when the implantable scaffold is in dangerous proximity to the vulnerable anatomical structures.

Furthermore, in the method disclosed herein, the scaffold designing engine selects 111 an optimal location for a fixation member, for example, a fixation screw, of an optimal length to be disposed in an optimal orientation to ensure secure anchoring of the fixation member to a neighboring bone tissue, while preventing protrusion of the fixation member beyond a necessary anchoring volume into a dangerous proximity to the identified vulnerable anatomical structures. The scaffold designing engine plans positioning of the fixation member safely beyond the dangerous proximity to the identified vulnerable anatomical structures. The scaffold designing engine renders 112 an enhanced two-dimensional (2D) outer surface of the implantable scaffold incorporating a pilot hole for the fixation member in the selected optimal location and the optimal orientation. In an exemplary embodiment, the scaffold designing engine selects the optimal location, the optimal length, and the optimal orientation of the fixation member using one or more of: deterministic algorithms, AI algorithms comprising deep learning algorithms, and interactive user input based on a visualization of an axis and a shape of the fixation member on the 3D image visualization and the complementary, cross-sectional image visualizations of the data volume. The scaffold designing engine identifies 113 a minimal proximity of one or more of the implantable scaffold and the fixation member to the vulnerable anatomical structures, and generates 114 an alert when the implantable scaffold and/or the fixation member are in dangerous proximity to the vulnerable anatomical structures. In an exemplary embodiment, the surface of any object including the implantable scaffold, the surface of the fixation member, and surfaces of the vulnerable anatomical structures are represented by a dense mesh of sampling points. For the purpose of assessment of the minimal distance between objects, for each test point in the mesh of one object, the scaffold designing engine calculates the distances to all points of the mesh on the other object as a distance from the mesh test point on one object to the other object, and identifies the minimal distance between the objects as the minimum of all distances from the multiplicity of test mesh points on one object to the other object. The scaffold designing engine then considers the minimum distance from all test mesh points of one object to the other object as the minimal proximity between these two objects. If the minimal proximity becomes less than the professionally accepted safe distance, the scaffold designing engine generates an alert, for example, through a message on the graphical user interface and an optional audible signal. The scaffold designing engine allows and incorporates 115 adjustments to the outer shape of the implantable scaffold and the location of the fixation member based on the alert.

Consider an example where a medical practitioner, for example, a physician, utilizes the scaffold designing engine to generate a “fully inhouse” design of an outer shape of an implantable scaffold. The scaffold designing engine acquires multiple cross-sectional medical diagnostic images fully covering a bone regeneration volume and generates a digital representation of the bone regeneration volume as a data volume. The scaffold designing engine renders four digital representations of the data volume including three complementary, cross-sectional image visualizations of the data volume, also referred to as planar views or planar cross-sectional sampling of the data volume, and one 3D image visualization of the data volume on a graphical user interface (GUI) to allow the physician to interact with the scaffold designing engine and execute a computer aided design (CAD) process for designing the outer shape of the implantable scaffold. The interactions of the physician with the scaffold designing engine via the GUI are herein referred to as “user interactions”. The physician interacts with the scaffold designing engine via the GUI as follows. The physician outlines an area of a bone defect on one of the four complementary, cross-sectional image visualizations of the data volume by drawing a three-dimensional (3D) defect contour such that all points of the 3D defect contour are substantially close to a bone surface. The accuracy to which the 3D defect contour should match the actual bone surface should be adequate for scaffolding and may be different for various surgical and dental applications.

The physician then defines a desired orientation for a flat base of the implantable scaffold through its normal vector, which is the direction of vertical walls of the implantable scaffold and specifies a desired location for the base of the implantable scaffold relative to the bone defect as a first base plane. On receiving the user interactions from the physician, the scaffold designing engine generates a mathematical digital representation of a tubular or cylindrical surface that completely contains the defect contour and whose axis is parallel to the normal vector as defined by the physician. This cylindrical surface is herein referred to as an “extrusion cylinder”. The scaffold designing engine then generates a mathematical digital representation of a first base plane of the implantable scaffold and identifies an area of the first base plane enclosed within a projection of the 3D defect contour onto the first base plane as a base facet of the implantable scaffold.

A canonic mathematical representation of the first base plane comprises, for example, a definition of: (1) 3D coordinates of either of its points called an origin point; and (2) a 3D normal vector perpendicular to the first base plane. In an exemplary implementation of the method disclosed herein, the normal vector of the first base plane is defined as the extrusion vector of the first tubular surface while a basepoint is selected by the following process:
(1) The defect contour is uniformly covered by a sequence of test points and each point is projected onto the extrusion vector;
(2) The outmost projection, that is in a direction outside of the bone defect, is taken as a first approximation of the basepoint;
(3) This first approximation is then protracted along the normal vector outside the bone surface by a distance equal to three-layer thicknesses of the implantable scaffold. This protracted location is taken as the origin point of the first base plane. Such selection of the basepoint ensures that at least the first three layers of the resulting implantable scaffold are fully inscribed into the “walls” of the implantable scaffold before shrinking in size and shape when entering into the area of the “dome” of the implantable scaffold.

The scaffold designing engine further generates a mathematical digital representation of a bone surface segment, enclosed by the 3D defect contour drawn by the physician. This bone surface segment is herein referred to as the “membrane”. The scaffold designing engine then generates a mathematical digital representation of the outer surface of the implantable scaffold by combining the above three surface segments, namely, (1) a segment of the base facet; (2) a segment of the membrane, herein referred to a “membrane segment”; and (3) a segment of the wall surface enclosed between the base facet and the membrane, herein referred to as a “wall segment”.

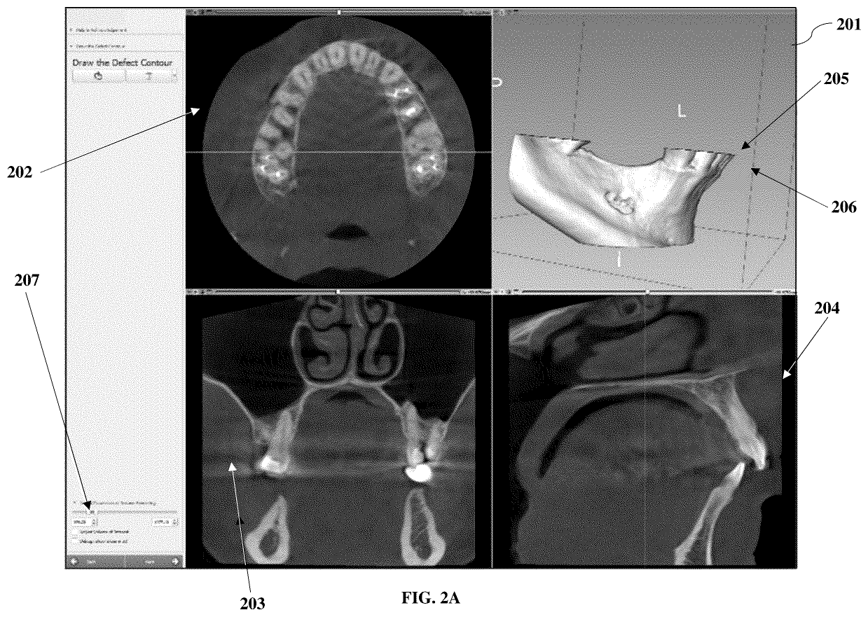

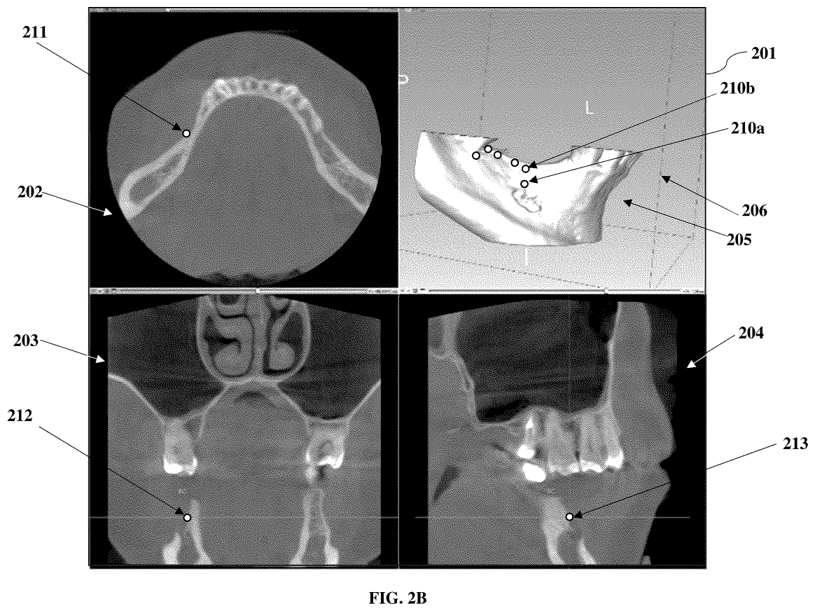

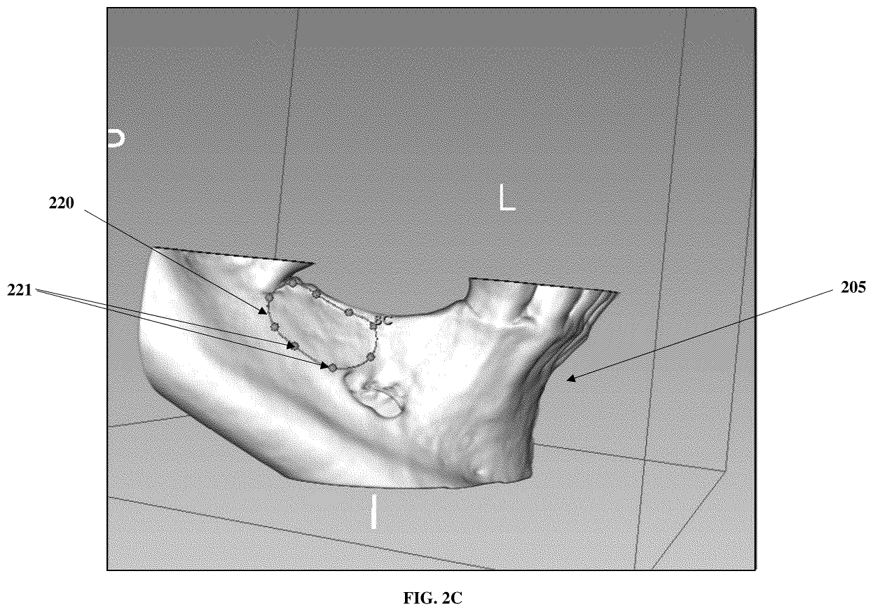

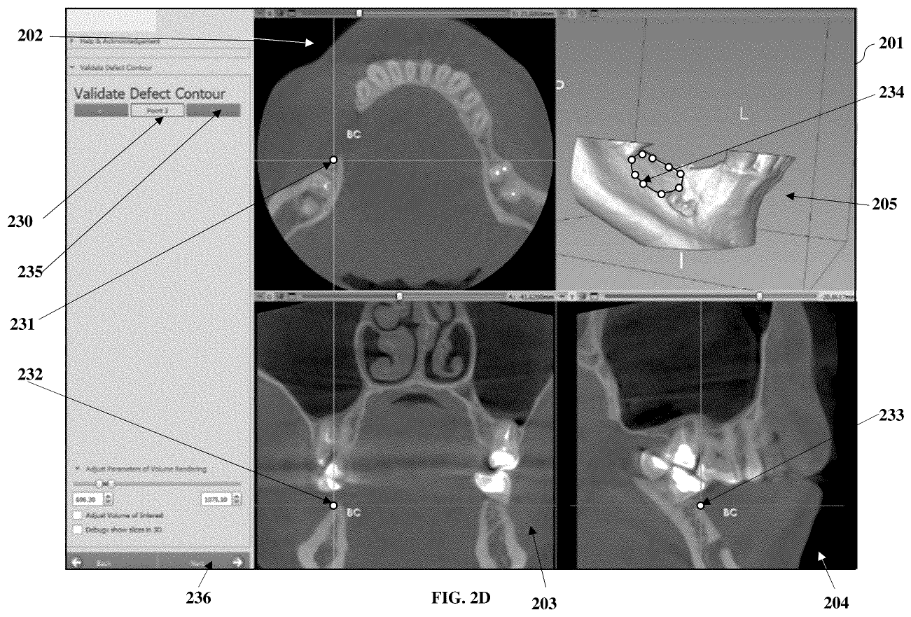

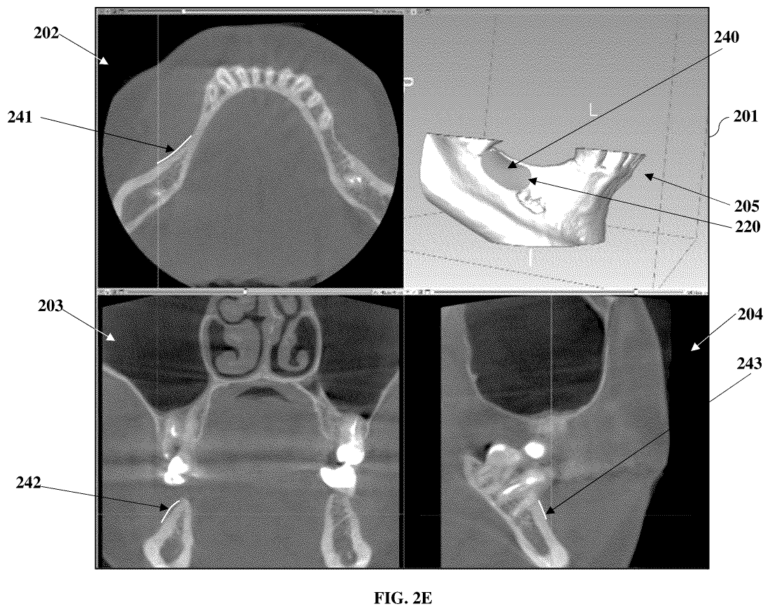

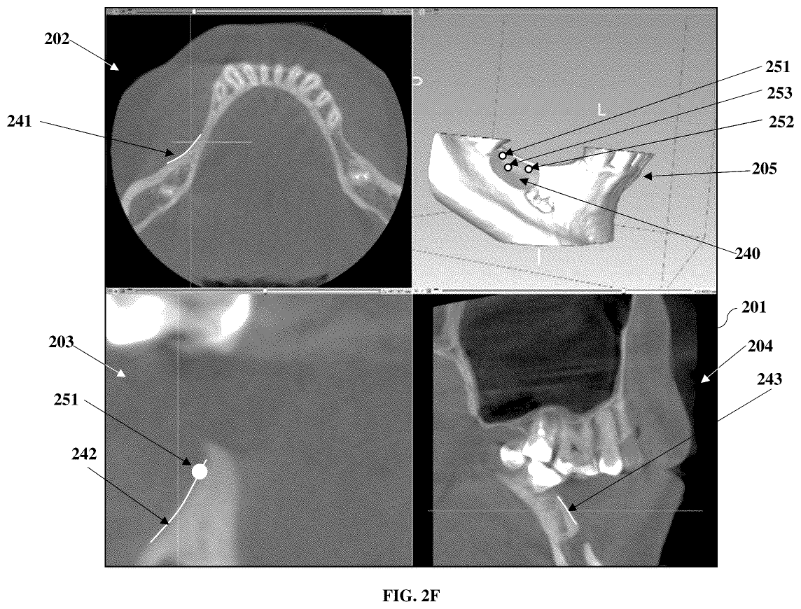

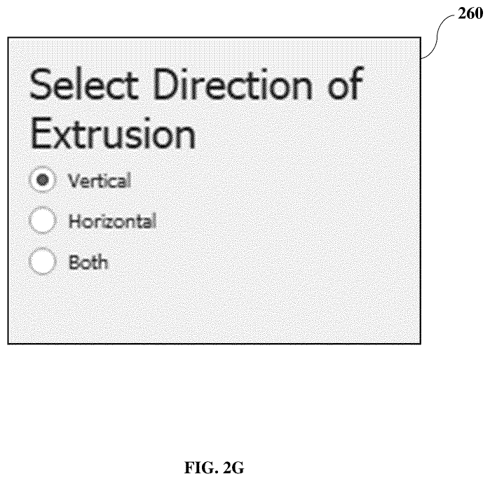

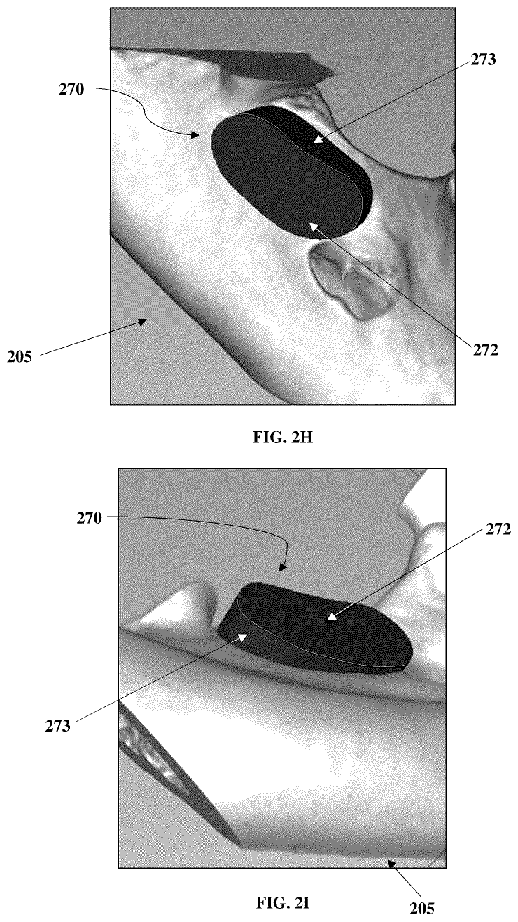

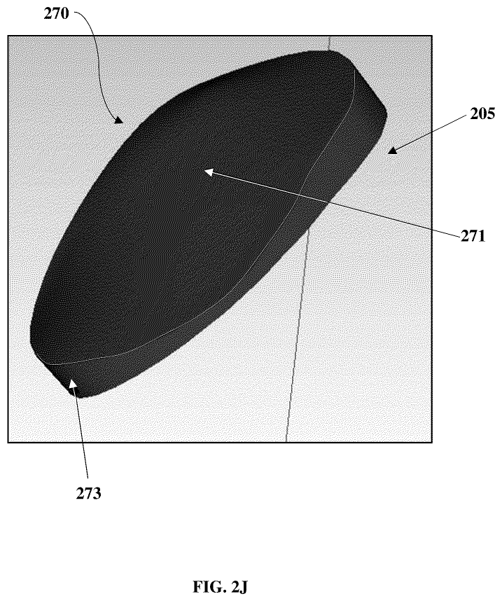

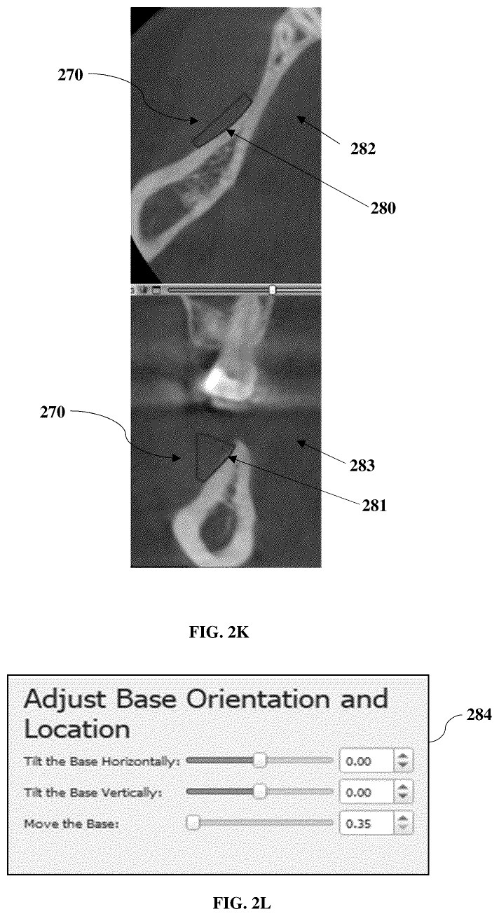

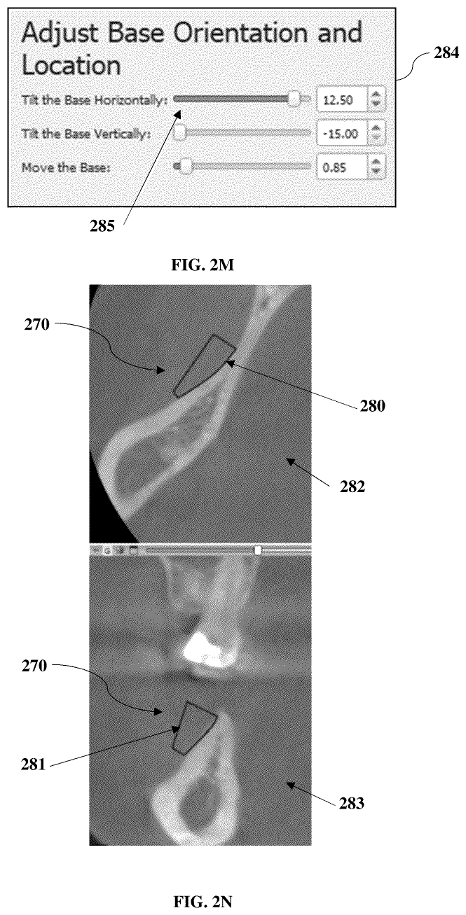

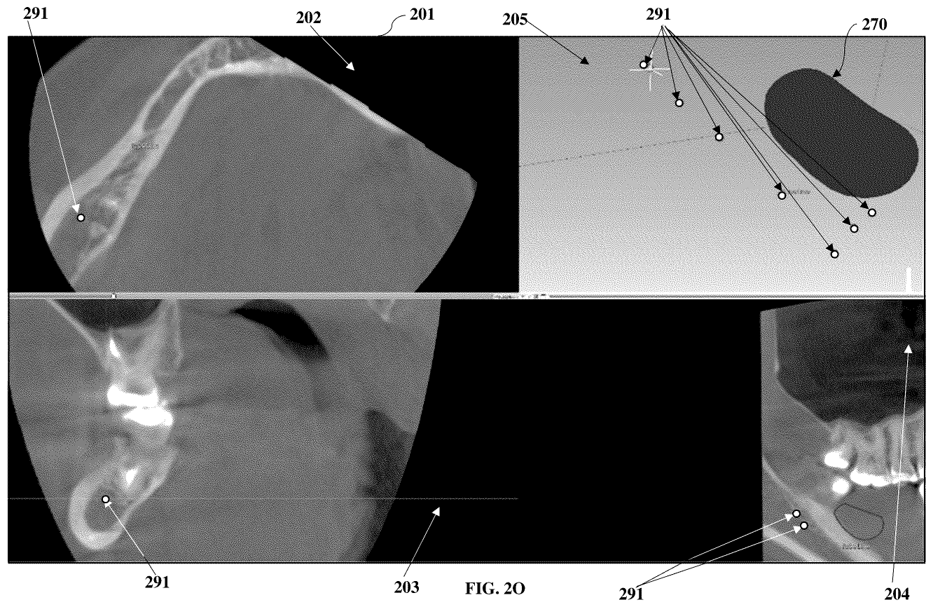

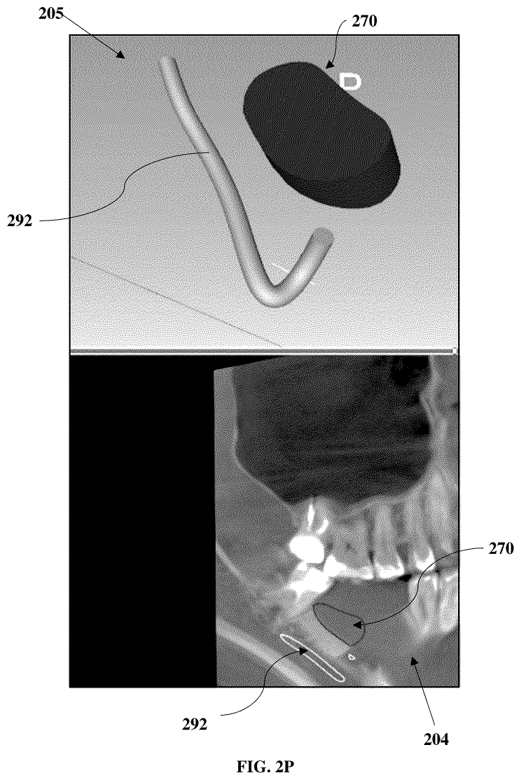

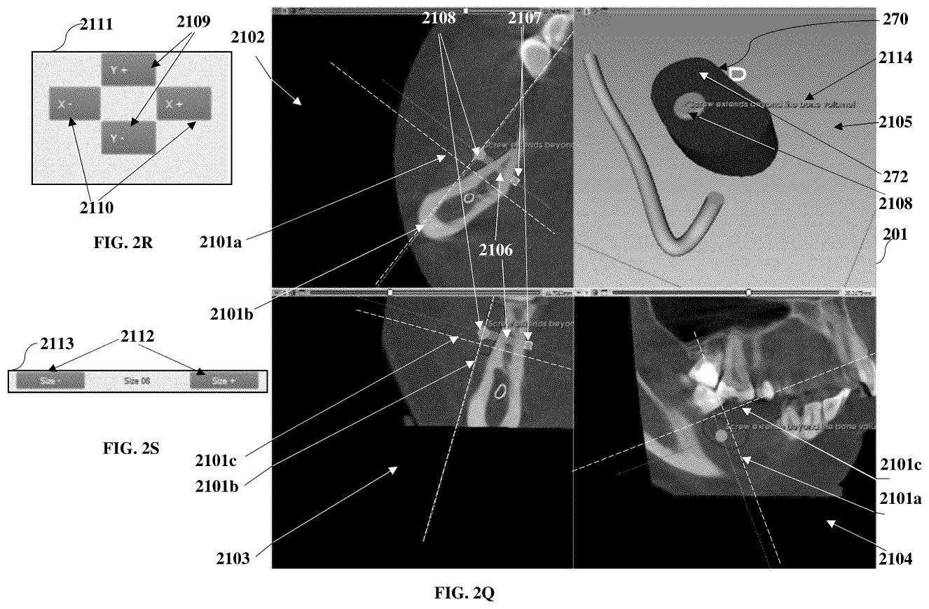

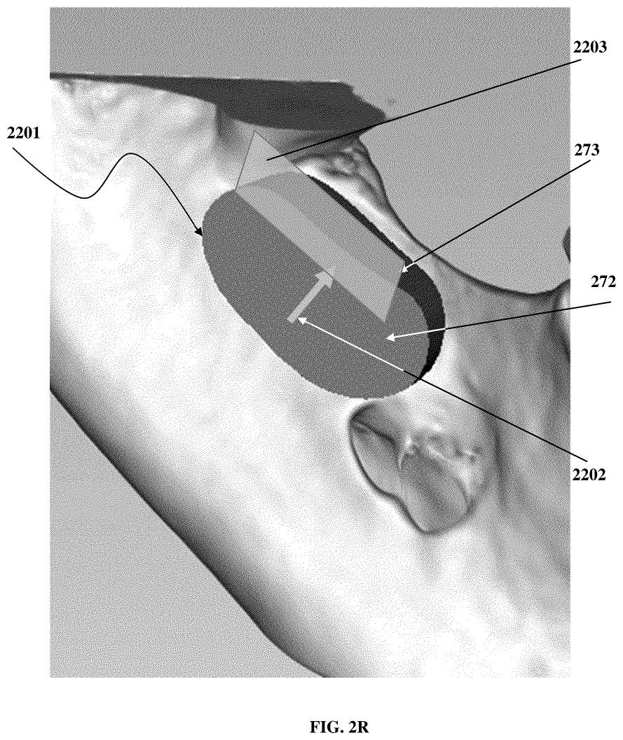

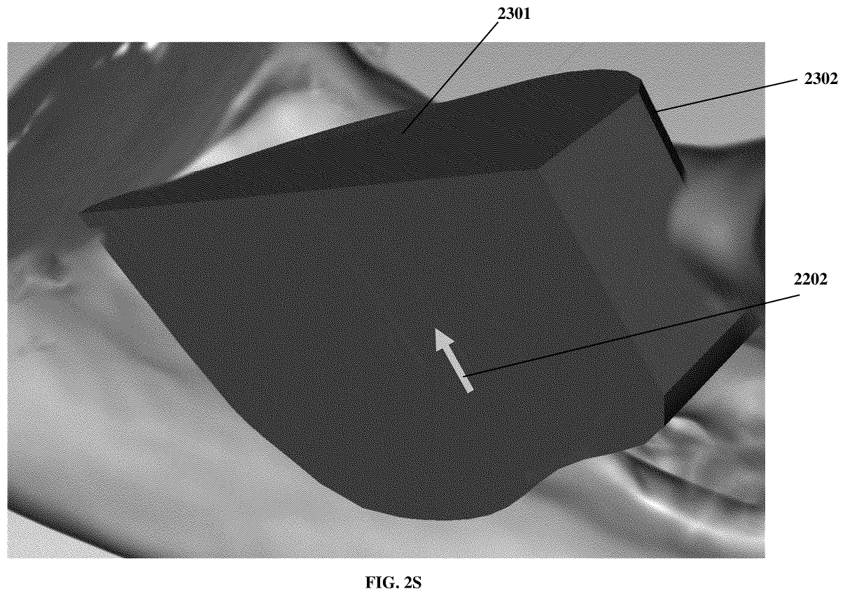

FIGS. 2A-2T illustrate screenshots of graphical user interfaces (GUIs) rendered by the scaffold designing engine for designing and planning fixation of an implantable scaffold. Consider another example where a user, for example, a physician, utilizes the scaffold designing engine to generate a “fully inhouse” design of an outer shape of an implantable scaffold. The user subjects a patient to imaging on a medical imaging device, for example, a computed tomography (CT) scanner or other suitable medical imaging device, capable of producing voxelated data within a volume substantially covering the volume for bone regeneration. The medical imaging device produces and outputs the voxelated data in any data interchange format that is readable by other conventional medical imaging apparatuses and that renders the voxelated data in multiple two-dimensional (2D) cross-sectional matrices accompanied by metadata attributing each of these 2D cross-sectional matrices to a specific patient, exam, spatial location, and spatial orientation. For example, the medical imaging device produces and outputs the voxelated data in a Digital Imaging and Communications in Medicine (DICOM) format, which renders the voxelated data in multiple 2D cross-sectional matrices accompanied by metadata attributing each of these 2D cross-sectional matrices to a specific patient, exam, spatial location, and spatial orientation. In this example, the user loads the voxelated data in the DICOM format into a separate computing system comprising the scaffold designing engine, which guides the user through a rigid, yet flexible workflow comprising the following exemplary steps. It should be clear to those skilled in the art that a specific implementation of these exemplary steps is disclosed for an exemplary purpose only and other implementations reaching the same final and/or intermediate goals also fit the spirit of the method and the system disclosed herein.

The scaffold designing engine renders four complementary visual representations of the voxelated data, comprising three complementary, cross-sectional image visualizations 202, 203, and 204 and one three-dimensional (3D) image visualization 205 on a graphical user interface (GUI) 201 as illustrated in FIG. 2A. The 3D image visualization 205 illustrates a digital representation of the bone regeneration volume as a data volume 206, for example, in the form of a data cube as illustrated in FIG. 2A. In an exemplary embodiment, the GUI 201 provides a visualization of the cross-sectional data in three stacks of planar images where the plane of each stack is perpendicular to a respective axis of an original DICOM volume as defined in the DICOM metadata. In an exemplary embodiment, the scaffold designing engine executes a fully automated or interactive, specialized embedded algorithm for generating a volumetric visualization of an optical nonuniform cloud based on the original voxelated data as disclosed in the description of FIG. 1A. The scaffold designing engine generates the volumetric visualization of the optical nonuniform cloud by attributing particular optical quantitative properties to each local point within the optical nonuniform cloud. The optical quantitative properties comprise, for example, local point opacity, point luminosity, and point light-scattering characteristics. In an exemplary embodiment, the optical quantitative properties are defined as a function of the respective voxel value of the original data volume. In another exemplary embodiment, the optical quantitative properties are defined as a generic mathematical function comprising local derivatives and integration over a surrounding vicinity.

In an exemplary embodiment as illustrated in FIG. 2A, the scaffold designing engine builds a 3D image visualization 205 of a bone surface through a simulative tracing of rays emitted by a remote light source, whether point-sized or transient, through the volumetric visualization of the optical nonuniform cloud onto a flat screen of the GUI 201. The scaffold designing engine renders tools, for example, slider strips 207, on the GUI 201 to allow the user to adjust parameters of the specialized embedded algorithm that converts local voxelated data into optical properties of the optical nonuniform cloud as a ramp function in such a way that the rendered 3D image provides the best and closest representation of the actual bone surface. One of the slider strips 207 allows the user to control upper and lower ramp values of a ramp function configured to convert the voxelated data into opacity of the optical nonuniform cloud. In an exemplary embodiment, the scaffold designing engine allows the user to drag the slider strip 207 as a solid body by its inner points, simultaneously adjusting upper and lower ramp values by the same amount, while dragging either end of the slider strip 207 adjusts only one upper/lower ramp value. The scaffold designing engine also renders adequate tools implemented, for example, as a mouse drag or a shift-drag over a 3D panel on the GUI 201, for allowing the user to adjust orientation of the remote light source and the data volume 206 relative to a ray-projected surface so that details and landmarks of the rendered image provide a best and optimal perception of a spatial structure, geometry, and borders of a bone defect and the surrounding healthy bone.

In an exemplary embodiment, the scaffold designing engine allows the user to outline the area of the bone defect by seeding an isolated point 210a over a perceived border of the bone defect on the 3D image visualization 205 of the bone surface as illustrated in FIG. 2B. Following the seeding, the specialized embedded algorithm generates the following output to the seeding:
(1) The user selects a desired location of the next seed point 210b on a projection plane of a 3D ray tracing visualization by moving a computer mouse pointer and indicates the selection by a mouse click. This newly seeded point 210b is visualized over the 3D image visualization 205 of the bone surface as a visual element as illustrated in FIG. 2B.
(2) The location of this newly seeded point 210b on the projection plane is then taken as an initial location of an imaginary test point, which is then gradually moved from a rendering screen, that is, the GUI 201, straight back to the light source through a gradual back projection by the specialized embedded algorithm.
(3) The scaffold designing engine employs a 3D visualization engine or a 3D point seeding engine to assess the visibility of the imaginary test point continuously upon its gradual back projection, and as soon as the moving imaginary test point dives into the volume of the optical nonuniform cloud into a marked optical depth and the visibility of the moving imaginary test point starts dropping fast, the scaffold designing engine stops the further back-projection of the moving imaginary test point.
(4) The scaffold designing engine employs the 3D visualization engine to then assess the final spatial location of the imaginary test point as a settlement of a new seed point on the bone surface and denotes current 3D coordinates of the new seed point as initial coordinates of a new contour seed point. The 3D visualization engine then visualizes the new contour seed point on the 3D image visualization 205. The 3D visualization engine then performs automatic scrolling of each of the opened cross-sectional image visualizations to the plane containing that contour seed point and visualizes that newly seeded contour seed point on a respective planar projection 211, 212, and 213 as illustrated in FIG. 2B.

The scaffold designing engine allows the user to interactively move the newly settled seed point on any of the three cross-sectional image visualizations 202, 203, and 204 or over the 3D image visualization 205, along with adjusting the orientation of the 3D image visualization 205, panning and zooming of any of the image visualizations 202, 203, 204, and 205, adjusting presentation parameters, to adjust conversion of local voxelated data into brightness of the respective area of the involved images. The adjustments allow the user to locate the newly settled seed point adjacent to what is professionally perceived as the bone surface. Upon spatial adjustment of the location of the seed point on any of the image visualizations 202, 203, 204, and 205, the scaffold designing engine scrolls other planar stacks accordingly to contain the new location of the seed point on all three cross-sectional image visualizations 202, 203, and 204. When the user perceives that the set of seed points provides an adequate representation of the defect contour 220, the user terminates the seeding process. In response to that termination, the scaffold designing engine executes the specialized embedded algorithm to build and visualize the full enclosed defect contour 220 with each individual seeding point 221 overlaid over the defect contour 220 as illustrated in FIG. 2C.

In an exemplary embodiment, the scaffold designing engine then prompts the user to inspect and confirm the spatial location of each seeded point one-by-one as illustrated in FIG. 2D. The scaffold designing engine instructs the user to validate and confirm the spatial location of each subsequent seeded point starting from the first seeded point using a control element 230 rendered on the GUI 201 as illustrated in FIG. 2D. Upon each step of the validation, the user assesses locations of each subsequent seeded point through its representations 231, 232, 233, and 234 presented on each of the four image visualizations 202, 203, 204, and 205 as illustrated in FIG. 2D, and either validates the location of each subsequent seeded point by hitting a “right arrow” button 235 on the GUI 201 or adjusts the location by dragging the visualized seeded point to a desired location, and then hits the “right arrow” button 235. When all the seeded points are validated, user hits the “Next” button 236 indicating his consent to locations of all seed points and to proceed to the next step of Cad workflow.

Upon this next step of the workflow the scaffold designing engine defines a membrane 240 as illustrated in FIG. 2E, through a three-dimensional (3D) interpolation of the defect contour 220 into its interior using standard methods of modern computer graphics. For example, the method of 3D interpolation simulates a spatial equilibrium of an elastic film tightly stretched over a rigid shape of the defect contour 220. In an exemplary embodiment, the 2D contiguous membrane 240 is configured to rest on an optical depth iso-surface of the optical nonuniform cloud.

The scaffold designing engine provides a visualization of the created membrane 240 on the 3D image visualization 205 and renders cross-sections 241, 242, and 243 of the membrane 240 on the cross-sectional image visualizations 202, 203, and 204, respectively, as illustrated in FIG. 2E, for examination by the user. The user may scroll through the stack of these cross-sectional image visualizations 202, 203, and 204 to ensure that the mathematically created digital model does not intrude into the bone volume, or otherwise does not leave a substantial gap between the membrane 240 and the surface of the bone defect. If the user is not satisfied about how accurately the inner area of the membrane 240 follows the bone surface, the user may adjust the geometry of the membrane 240 using dedicated tools provided by the scaffold designing engine. For example, by the user's discretion, the user may add anchor points 251, 252, and 253 over the surface of the membrane 240 using any of the four image visualizations 202, 203, 204, and 205 as illustrated in FIG. 2F, and interactively move the anchor points 251, 252, and 253 into the bone surface or away from the bone surface using the click-and-drag mouse operations to achieve the desired tight fit between the membrane 240 and the bone surface, or in an exemplary embodiment, to ensure a desired gap between the membrane 240 and the bone surface within a desired area.

Upon seeding and spatial adjustment of each subsequent anchor point, the scaffold designing engine rebuilds the shape of the membrane 240 in such a way that membrane 240 includes the newly adjusted anchor point and all other previously added anchor points as well as all points of the defect contour 220 illustrated in FIG. 2C. The scaffold designing engine provides a visualization of the added anchor points 251, 252, and 253 on the 3D image visualization 205 as illustrated in FIG. 2F. The scaffold designing engine allows the user to select any of these anchor points 251, 252, and 253 and automatically scrolls all opened cross-sectional image visualizations to provide a visualization of the selected anchor point. The scaffold designing engine allows the user to adjust the spatial location of the selected anchor point using any of the opened cross-sectional image visualizations 202, 203, and 204 and the 3D visualization 205. Following the adjustments made by the user, the scaffold designing engine readjusts the digital representation of the membrane 240 to adhere to the new location of the selected anchor point. The scaffold designing engine allows the user to continue adjusting the anchor points until the user is satisfied with the view of the membrane 240 and the shape of its cross-sections 241, 242, and 243.

The scaffold designing engine renders a panel 260 as illustrated in FIG. 2G to allow the user to define a desired major direction of extrusion, for example, vertical, or horizontal, or both vertical and horizontal. The scaffold designing engine then identifies a long axis of the defect contour 220, for example, as a segment connecting two most distant points on the defect contour 220 and derives an extrusion vector depending on the user's selection on the panel 260. If the user selects a horizontal extrusion, then the long axis is first projected on a horizontal plane and then rotated by 90 degrees on that horizontal plane. Alternatively, if the user selects a vertical extrusion, then the extrusion vector is set straight vertically. The scaffold designing engine then identifies an area or a location of the first base plane which would become the location of the first printable layer of the implantable scaffold, as a base facet. The first base plane is an extension of the base facet. In an exemplary embodiment, the scaffold designing engine identifies the base facet as follows:
(1) the scaffold designing engine projects the defect contour 220 onto the extrusion vector;
(2) the scaffold designing engine identifies the most forward point of this projection of the defect contour 220 in the direction of the extrusion vector; and (3) the scaffold designing engine moves the identified point along a positive direction of the extrusion vector by a technological offset of, for example, about 0.5 millimeter (mm) to about 2 mm. The scaffold designing engine considers the identified point as the location or the origin point of the first base plane, while defining the orientation of the first base plane as orthogonal to the extrusion vector. The scaffold designing engine then executes dedicated mathematical algorithms implemented in computer aided design (CAD) software to perform the following:
(1) Project the spatial 3D defect contour 220 onto the previously defined first base plane as a base contour.
(2) Identify the area of the first base plane circumferenced by the base contour, herein referred to as the base facet, that is, the flat base of the future implantable scaffold, and create a mathematical digital model of the base facet;
(3) Create a mathematical digital model of the tubular or cylindrical surface, also referred to as the “extrusion cylinder”, that completely contains the defect contour 220 and the base contour as two separate circumferences and whose axis is parallel to a normal vector of the first base plane;
(4) Create a mathematical digital model of a segment of the extrusion cylinder enclosed between the base contour and defect contour 220. The segment of the extrusion cylinder is herein referred to as the “wall segment”; and
(5) Create a mathematical digital model of a surface envelope or the outer surface of the implantable scaffold, as a contiguous joinder of three airtight-connected surface segments: the base facet, the wall segment, and the membrane segment.

The scaffold designing engine provides a visualization of the mathematical digital model of the outer surface of the implantable scaffold 270 on the 3D image visualization 205 as illustrated in FIGS. 2H-2J for inspection by the user from any point of view. The outer surface of the implantable scaffold 270 represents the outer shape of the implantable scaffold 270. The base facet 272, the wall segment 273, and the membrane segment 271 are illustrated in the 3D image visualization 205 shown in FIGS. 2H-2J. The scaffold designing engine allows the user to inspect the membrane segment 271 by removing the rendering of the bone from visualization and properly orienting the 3D image visualization 205 illustrated in FIG. 2J.

The scaffold designing engine renders respective cross-sections of the implantable scaffold 270 on the cross-sectional image visualizations 282 and 283 as illustrated in FIG. 2K and FIG. 2N for examination by the user. FIG. 2K indicates the outer shape 280, 281 of the implantable scaffold 270 in different views. If the user is not satisfied with the outer shape 280, 281 of the implantable scaffold 270 automatically generated by the scaffold designing engine, the scaffold designing engine allows the user to adjust the location and orientation of the first base plane according to the user's professional preferences using a panel 284 as illustrated in FIG. 2L. On receiving adjustments 285 from the user via the panel 284 as illustrated in FIG. 2M, the scaffold designing engine regenerates the outer shape 280, 281 of the implantable scaffold 270 as illustrated in FIG. 2N, for reexamination by the user. When the user is satisfied with the location and the spatial geometry of the outer shape 280, 281 of the implantable scaffold 270 regenerated by the scaffold designing engine based on the adjustments 285 made using control elements on the panel 284 illustrated in FIG. 2M, the user may proceed to identify vulnerable anatomical structures, for example, a mandible nerve, located in the vicinity of the implantable scaffold 270 for planning the fixation of the implantable scaffold 270 as disclosed in the description of FIG. 1B.

In an example, the scaffold designing engine outlines the mandible nerve through seeding of seed points 291 along its axis as illustrated in FIG. 2O. The scaffold designing engine allows the user to interactively perform seeding on the 3D image visualization 205 and on the cross-sectional image visualizations 204, 202, and 203 as illustrated in FIG. 2O, similar to the process of seeding points along the defect contour 220 disclosed above. When the user indicates satisfaction with the tracing of the seed points 291 on all four image visualizations 202, 203, 204, and 205 on the GUI 201 illustrated in FIG. 2O, the scaffold designing engine renders the 3D curved axis of the mandible nerve through a 3D interpolation between seeded points similarly to interpolation of the bone defect contour between its seed points and then renders a nerve tube as a “curved flexible cylinder of desired radius built around such produced 3D curved axis and provides a visualization of a whole mandible nerve tube 292 with respect to the implantable scaffold 270 and/or bone shapes on the 3D image visualization 205 and the cross-sectional image visualization 204 as illustrated in FIG. 2P. The scaffold designing engine then allows the user to readjust the seed points 291 along the nerve axis, and/or add new seed points, and/or remove some of the existing seed points 291. The scaffold designing engine also allows the user to adjust a radius of a representative volume of the mandible nerve. When the user completes the definition of the mandible nerve, the scaffold designing engine assesses the closest proximity of the implantable scaffold 270 to the mandible nerve tube 292 and in an exemplary embodiment, and issues an alert or a warning if the implantable scaffold 270 comes in dangerous proximity to the mandible nerve tube 292. The scaffold designing engine performs the closest proximity assessment, for example, by covering the surface of the implantable scaffold 270 and the surface of the mandible nerve tube 292 with a sufficiently dense mesh of points and calculating the distance between each pair of points—one on the implantable scaffold 270 and another on the mandible nerve tube 292, and then taking the minimal distance of all. If the closest proximity becomes less than the professionally accepted safe distance, the scaffold designing engine generates an alert, for example, through a message on the graphical user interface and an optional audible signal. The scaffold designing engine then allows the user to adjust or redesign the outer shape of the implantable scaffold 270 to avoid such dangerous proximity.

On identifying the vulnerable anatomical structures, for example, a mandible nerve, that should be avoided, the scaffold designing engine initiates the planning of a fixation member, for example, a fixation screw, and a pivot hole. The scaffold designing engine allows the user to select a location, an orientation, and a length for the fixation screw. For example, the user may select a fixation screw of size 6 or size 8 or another length from a predefined library to be fixed at the geometrical center of the implantable scaffold 270 and to be oriented substantially perpendicular to the implantable scaffold 270. Based on the user's selections, the scaffold designing engine plans the geometry of the pilot hole to be shaped during a process of 3D printing of the implantable scaffold 270.

If the user is satisfied with the relative location of the implantable scaffold 270 versus the mandible nerve tube 292, the user proceeds to define a location, an orientation, and a length for the fixation screw. In an exemplary embodiment, the scaffold designing engine readjusts the orientations of the cross-sectional image visualizations 2102, 2103, and 2104 illustrated by dashed cross-hair lines 2101a, 2101b, and 2101c into new orientations for an optimal visualization of the cross-section of the fixation screw 2106 illustrated in FIG. 2Q, in relation to the implantable scaffold body and surrounding anatomical tissues. For example, the scaffold designing engine sets a first cross-sectional image visualization 2104 illustrated by a dashed cross-hair line 2101b parallel to the base facet 272 of the implantable scaffold 270, and sets the other two cross-sectional image visualizations 2102 and 2103 illustrated by the dashed cross-hair lines 2101c and 2101a, respectively, in FIG. 2Q.

In an exemplary embodiment, the scaffold designing engine sets an initial position of a head 2108 of the fixation screw 2106 to a geometrical center of the base facet 272 and sets a direction of a body of the fixation screw 2106 normal to the base facet 272 as illustrated on FIG. 2Q. The scaffold designing engine provides a visualization of an axial cross-section of the fixation screw 2106 on respective cross-sectional image visualization 2102, 2103, and 2104 and on a 3D image visualization 2105 in relation to the body of the implantable scaffold 270 rendered in solid color and anatomical tissues rendered according to local tissue density as illustrated in FIG. 2Q. The scaffold designing engine allows the user to adjust the location and the direction of the fixation screw 2106 as illustrated on FIG. 2Q. In an example, the user adjusts the location of the fixation screw 2106 through the use of the up and down buttons 2109 and the left and right buttons 2110 on a panel 2111 rendered by the scaffold designing engine as illustrated in FIG. 2R, which allows the user to move the fixation screw 2106 in a self-parallel fashion such that the head 2108 of the fixation screw 2106 is moved in small discrete steps over the base facet 272 of the implantable scaffold 270, while the direction of the fixation screw 2106 remains the same. The scaffold designing engine allows for an alternative method for interactively adjusting both location and orientation of the fixation screw 2106 through dragging by anchor points that are visualized on the cross-sectional image visualizations 2102 and 2103, when the user scrolls an appropriate planar stack to an image containing the central axis of the fixation screw 2106. The scaffold designing engine provides a visualization of one anchor point at the head 2108 of the fixation screw 2106 and another anchor point on a tip 2107 of the fixation screw 2106 in the cross-sectional image visualizations 2102 and 2103 illustrated in FIG. 2Q. Dragging the anchor point at the head 2108 of the fixation screw 2106 causes a relocation of the fixation screw 2106 in a self-parallel fashion. Dragging the anchor point at the tip 2107 of the fixation screw 2106 changes the orientation of the fixation screw 2106 so that the tip of the screw follows the relocated point 2107, while keeping the position of its head 2108 steady. When orientation of the fixation screw 2106 is changed, the scaffold designing engine rebuilds the cross-sectional image visualizations 2102, 2103, and 2104 used for the visualization of the fixation screw 2106 in such a way that one cross-sectional image visualization 2104 remains perpendicular to the axis of the fixation screw 2106, while other two cross-sectional image visualizations 2102 and 2103 contain the axis of the fixation screw 2106 in-plane.

The scaffold designing engine also allows the user to adjust a length of the fixation screw 2106. For example, by activating Size+/Size− buttons 2112 on a panel 2113 rendered by the scaffold designing engine as illustrated in FIG. 2Q, the user selects the next larger or next smaller commercially available length for the fixation screw 2106 according to standard screw catalogs. In another example, dragging the anchor point on the tip 2107 of the fixation screw 2106 in the direction of the fixation screw 2106 also adjusts the length of the fixation screw 2106 in a desired direction according to the closest commercially available lengths in standard screw catalogs. Upon any adjustment of the position or the length of the fixation screw 2106, the scaffold designing engine assesses the closest proximity of the fixation screw 2106 to the previously identified vulnerable anatomical structures. In an exemplary embodiment, the scaffold designing engine performs the proximity assessment by covering the surface of the fixation screw 2106 and the surface of the vulnerable anatomical structures with a sufficiently dense mesh of points and calculating the distance between each pair of points - one on the fixation screw 2106 and the other on the vulnerable anatomical structures, and then taking the minimal distance of all the points. If the proximity becomes dangerously small, the scaffold designing engine generates and issues an alert or a warning to the user and blocks the adjustment. For example, the scaffold designing engine issues alert with a warning message “The fixation screw is dangerously close to the nerve”. Upon any adjustments of the length of the fixation screw 2106, the scaffold designing engine executes an algorithm using, for example, a deep learning AI model trained on multiple training datasets, to determine whether the body of the fixation screw 2106 including its tip 2107 is entirely embedded within the mandible bone. By executing the algorithm, if the scaffold designing engine identifies that the tip 2107 of the fixation screw 2601 extends beyond the mandible bone, the scaffold designing engine generates and issues an audio alert and activates a warning message 2114, for example, “Screw is beyond the bone volume”, on the image visualizations 2102, 2103, 2104, and 2105 of the GUI 201 as illustrated in FIG. 2Q.

Enumerated below are additional exemplary embodiments of the method for designing an outer shape of an implantable scaffold. In an exemplary embodiment, the scaffold designing engine subjects medical imaging data of the acquired cross-sectional medical diagnostic images to preprocessing for eliminating or substantially reducing noise and artifacts of the voxelated data and/or for enhancing specific features and characteristics of the voxelated data. In another exemplary embodiment, the optical characteristic of the volumetric visualization of the optical nonuniform cloud, also referred to as a “cloud model”, used for simulating a 3D view is a mathematical function defined over voxelated data in a data volume. In this exemplary embodiment, the mathematical function comprises a combination of local values, gradients, and derivatives as well as averages or other integral characteristics defined over a vicinity of each volume point inside the data volume. An example of the mathematical function is a ramp function constructed in such a way that all values below a so-called “floor value” is mapped to zero, all values above a so-called “ceiling value” is mapped to one thousand, while all values between the floor value and the ceiling value are mapped in between 0 and 1000 through the linear or “S” curve. In another exemplary embodiment, the 3D view is a simulated surface, herein referred to as an “iso-surface”, representing a constant value of the original data volume. In another exemplary embodiment, the 3D view is a simulated surface representing particular topological features of a mathematical function, for example, the above-disclosed ramp function, built over voxelated data. The topological features comprise, for example, crests and troughs, surfaces of maximum gradient, iso-surfaces, and other mathematically defined features. In another exemplary embodiment, the scaffold designing engine generates the 3D view by executing any deterministic algorithm or AI algorithm comprising, for example, neural networks and/or deep learning algorithms such as standard noise reduction AI algorithms.

In another exemplary embodiment, the defect contour 220 is set through a continuous drawing implemented in a single stroke or as a series of multiple continuous strokes. In another exemplary embodiment, the walls of the implantable scaffold connecting the base facet with the bone membrane is not a segment of a cylindrical surface but represents a more complicated first tubular surface. The shape of the first tubular surface allows printing of the implantable scaffold on an extrusion 3D printer. In another exemplary embodiment, the scaffold designing engine allows the user to construct a more complex surface than a flat base facet and connecting walls, over the bone membrane surface, provided this complex surface is printable on an extrusion 3D printer with temporary supports or on other non-extrusion 3D printer capable of producing a bone regenerating, implantable scaffold.

In another exemplary embodiment, the scaffold designing engine identifies the location of the fixation screw 2106 and its length according to predefined anchoring characteristics of the mandible bone. Ideal anchoring of the fixation screw 2106 is achieved when the fixation screw 2106, upon its exit beyond the volume of the implantable scaffold, first penetrates the buckle or lingual cortical shell of the mandible bone, penetrates the volume filled with the marrow bone, and rests there without reaching the opposite cortical shell. This ideal situation is not possible for certain locations of the head 2108 of the fixation screw 2106, as at that location, the cross-section of the mandible bone contains only the cortical bone without featuring any marrow volume.

FIGS. 3A-3E illustrate complex geometries of individual linear struts and bars encapsulated by the implantable scaffold. The scaffold designing engine designs the outer shape of the implantable scaffold to encapsulate the complex geometries of individual linear struts and bars separated by air-filled gaps as illustrated in FIGS. 3A-3E, which in an exemplary embodiment, provides a porous structure required for initial placement of bone grafting material and for further ingrowth of new bone and a vascular and nerve network. In an example, the complex geometry of individual linear struts comprises a succession of layers, where each layer comprises equidistant parallel bars with connectors so that the overall layer visually resembles a straight line compressed into a snake-like geometry as illustrated in FIGS. 3A-3E. Directions of the parallel bars in each subsequent layer is substantially perpendicular to the previous layer. Consider an example where an external volume of the implantable scaffold is of a cubical shape. The scaffold designing engine dissects this external volume into a layers of a square shape as illustrated in FIGS. 3A-3B. The desired, 3D-printable, highly porous structure of the implantable scaffold is produced by continuously extruding a polymer bead in a snake-like geometry starting from one corner of a square and ending up in a diametral corner of the same layer, through an extruder nozzle of an extrusion 3D printer. In an exemplary embodiment, the snake-like geometry 301 comprises long runs aligned in an X-direction and short connecting runs in a Y-direction as illustrated in the left panel of the FIG. 3A. In another exemplary embodiment, the snake-like geometry 301 comprises long runs in the Y-direction and short connecting runs in the X-direction as illustrated in the right panel or the FIG. 3A. The extruder nozzle is lifted up by the thickness of one layer and the similar snake-like geometry is extruded over the previous layer; however, this snake-like geometry of the top layer comprises long runs aligned in the other direction than the long runs of the underlying layer as illustrated in FIG. 3B. In this configuration, the long runs of consecutive layers are interlocked and these two layers become mechanically welded to each other as a newly extruded bead solidifies over an already cooled-down bead of the previous layer in intersection points. Consider another example where an external volume of the implantable scaffold is of a non-rectangular outer shape. The internal geometry of the consecutively extruded layers in this example are illustrated in FIGS. 3C-3D, while the resulting double-layer conglomerate 302 is illustrated in FIG. 3E.

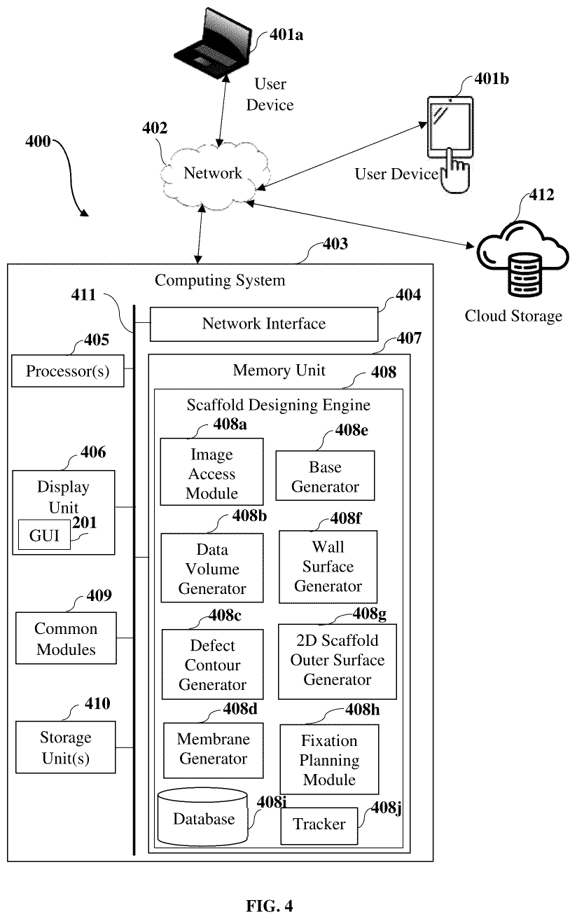

FIG. 4 illustrates an architectural block diagram of an exemplary implementation of a system 400 comprising the scaffold designing engine 408 for designing and planning fixation of an implantable scaffold. The scaffold designing engine 408 is configured to design the implantable scaffold in a seamless workflow tuned to specifics of a geometrical design of bone regeneration implants. In an exemplary embodiment as illustrated in FIG. 4, the scaffold designing engine 408 is deployed in a computing system 403 without networking. The computing system 403 is programmable using high-level computer programming languages. The computing system 403 is an electronic device, for example, one or more of a personal computer, a tablet computing device, a laptop, a notebook, a workstation, a client device, one or more servers, a network-enabled computing device, an interactive network-enabled communication device, any other suitable computing equipment, combinations of multiple pieces of computing equipment, etc.

In another exemplary embodiment, the scaffold designing engine 408 is implemented in a cloud computing environment via a network 402. As used herein, “cloud computing environment” refers to a processing environment comprising configurable, computing, physical and logical resources, for example, networks, servers, storage media, virtual machines, applications, services, etc., and data distributed over the network 402. The cloud computing environment provides an on-demand network access to a shared pool of the configurable computing physical and logical resources. The scaffold designing engine 408 operates in the cloud computing environment comprising a cloud storage 412 configured to store and access a complete set of volumetric cross-sectional computer tomography (CT) or cone beam computed tomography (CBCT) images of a desired anatomical area, which are acquired in a Digital Imaging and Communications in Medicine (DICOM) format. In an exemplary embodiment, the scaffold designing engine 408 is a cloud-based platform implemented as a service for designing and planning fixation of an implantable scaffold. For example, the scaffold designing engine 408 is configured as a software as a service (SaaS) platform or a cloud-based software as a service (CSaaS) platform that automatically or interactively designs and plans fixation of the implantable scaffold. In an exemplary embodiment, the scaffold designing engine 408 is configured on a server or a network of servers in a cloud computing platform, for example, the Amazon Web Services (AWS®) platform of Amazon Technologies, Inc., the Micro® oft Azure® platform of Microsoft Corporation, etc. Each server is responsible for a full workflow of scaffold designing or, alternatively, a particular portion of the scaffold designing engine procedures and functions as backend enablers of the scaffold designing engine 408. In another exemplary embodiment, the scaffold designing engine 408 is implemented locally as an on-premise platform comprising on-premise software installed and run on client systems on the premises of an organization, for example, a dental laboratory, a dental office, etc., to meet privacy and security requirements.

The computing system 403 comprising multiple modules, for example, 408a to 408j, of the scaffold designing engine 408 are accessible to users, for example, a dental staff member, dental practitioners, etc., through a broad spectrum of technologies and user devices 401a and 401b such as personal computers, laptops, internet-enabled cellular phones, smartphones, tablet computing devices, etc., with access to the internet. The scaffold designing engine 408 integrates with an existing workflow seamlessly to automatically pull cross-sectional medical diagnostic images into the scaffold designing engine 408. The scaffold designing engine 408 is in operable communication with one or more user devices 401a and 401b via the network 402. The user devices 401a and 401b are electronic devices, for example, personal computers, tablet computing devices, mobile computers, mobile phones, smartphones, portable computing devices, laptops, personal digital assistants, workstations, client devices, portable electronic devices, network-enabled computing devices, interactive network-enabled communication devices, web browsers, portable media players, any other suitable computing equipment, combinations of multiple pieces of computing equipment, etc.

The network 402 is a short-range network or a long-range network, for example, one of the internet, satellite internet, an intranet, a wired network, a wireless network, a communication network that implements Bluetooth® of Bluetooth Sig, Inc., a network that implements Wi-Fi® of Wi-Fi Alliance Corporation, an ultra-wideband (UWB) communication network, a wireless universal serial bus (USB) communication network, a communication network that implements ZigBee® of ZigBee Alliance Corporation, a general packet radio service (GPRS) network, a mobile telecommunication network such as a global system for mobile (GSM) communications network, a code division multiple access (CDMA) network, a third generation (3G) mobile communication network, a fourth generation (4G) mobile communication network, a fifth generation (5G) mobile communication network, a long-term evolution (LTE) mobile communication network, a public telephone network, etc., a local area network, a wide area network, an internet connection network, an infrared communication network, etc., or a network formed from any combination of these networks. The scaffold designing engine 408 interfaces with the user devices 401a and 401b, and in an exemplary embodiment, with one or more database systems (not shown) and servers (not shown) to implement a scaffold designing and fixation planning service, and therefore more than one specifically programmed computing system is used for implementing the scaffold designing and fixation planning service.

In an exemplary embodiment, the scaffold designing engine 408 is deployed and implemented in the computing system 403 using programmed and purposeful hardware as illustrated in FIG. 4. In an exemplary embodiment, the scaffold designing engine 408 is a computer-embeddable system that designs an outer shape of an implantable scaffold and plans fixation of the implantable scaffold. As illustrated in FIG. 4, the computing system 403 comprises a non-transitory, computer-readable storage medium, for example, a memory unit 407, for storing computer program instructions defined by modules, for example, 408a, 408b, 408c, 408d, 408e, 408f, 408g, 408h, 408i, 408j, etc., of the scaffold designing engine 408. As used herein, “non-transitory, computer-readable storage medium” refers to all computer-readable media that contain and store computer programs and data. Examples of the computer-readable media comprise hard drives, solid state drives, optical discs or magnetic disks, memory chips, a read-only memory (ROM), a register memory, a processor cache, a random-access memory (RAM), etc. The computing system 403 further comprises at least one processor 405 operably and communicatively coupled to the memory unit 407 for executing the computer program instructions defined by the modules, for example, 408a to 408j, of the scaffold designing engine 408. The memory unit 407 is a storage unit used for recording, storing, and reproducing data, program instructions, and applications. In an exemplary embodiment, the memory unit 407 comprises a random-access memory (RAM) or another type of dynamic storage unit that serves as a read and write internal memory and provides short-term or temporary storage for information and instructions executable by the processor(s) 405. The memory unit 407 also stores temporary variables and other intermediate information used during execution of the instructions by the processor(s) 405. In another exemplary embodiment, the memory unit 407 further comprises a read-only memory (ROM) or another type of static storage unit that stores firmware, static information, and instructions for execution by the processor(s) 405.

In an exemplary embodiment, the modules, for example, 408a to 408j, of the scaffold designing engine 408 are stored in the memory unit 407 of the computing system 403. For purposes of illustration, the scaffold designing engine 408 is exemplarily shown to be a part of an in-memory system of the computing system 403; however, the scope of the system 400 disclosed herein is not limited to the scaffold designing engine 408 being part of an in-memory system, but extends to the scaffold designing engine 408 being distributed across a cluster of multiple computer systems, for example, computers, servers, virtual machines, containers, nodes, etc., coupled to the network 402, where the computer systems operate as a team and coherently communicate and coordinate with each other to share resources, distribute workload, and execute different portions of the logic to implement scaffold designing and fixation planning functions as a service. Each computer system in the cluster executes a part of the logic and coordinates with other computer systems in the cluster to provide the complete functionality of the system 400 and the method disclosed herein.

The processor(s) 405 in the computing system 403 is configured to execute the modules, for example, 408a to 408j, of the scaffold designing engine 408 for designing an outer shape of an implantable scaffold and planning fixation of the implantable scaffold. The scaffold designing engine 408, when loaded into the memory unit 407 and executed by the processor(s) 405, transforms the computing system 403 into a specially-programmed, special purpose computing device configured to implement the scaffold designing and fixation planning functionality disclosed herein. The processor(s) 405 refers to one or more microprocessors, central processing unit (CPU) devices, finite state machines, computers, microcontrollers, digital signal processors, logic, a logic device, an application specific integrated circuit (ASIC), a field-programmable gate array (FPGA), a chip, etc., or any combination thereof, capable of executing computer programs or a series of commands, instructions, or state transitions. In an exemplary embodiment, the processor(s) 405 is implemented as a processor set comprising, for example, a programmed microprocessor and a math or graphics co-processor. The scaffold designing engine 408 is not limited to employing the processor(s) 405. In an exemplary embodiment, the scaffold designing engine 408 employs a controller or a microcontroller.

Also illustrated in FIG. 4, is a data bus 411, a display unit 406, a network interface 404, and common modules 409 of the computing system 403. The data bus 411 permits communications and exchange of data between the components, for example, 404, 405, 406, 407, 409, and 410 of the computing system 403. The data bus 411 transfers data to and from the memory unit 407 and into or out of the processor(s) 405. The display unit 406, via a graphical user interface (GUI) 201, displays user interface elements such as input fields for allowing, for example, a user of the computing system 403 to input data and provide user interactions into the scaffold designing engine 408. The GUI 201 comprises, for example, any one of an online web interface, a web-based downloadable application interface, a mobile-based downloadable application interface, etc.

The network interface 404 is configured to connect the computing system 403 to the network 402. The network interface 404 is, for example, one or more of infrared interfaces, interfaces implementing Wi-Fi® of Wi-Fi Alliance Corporation, universal serial bus (USB) interfaces, Ethernet interfaces, frame relay interfaces, cable interfaces, digital subscriber line interfaces, token ring interfaces, peripheral component interconnect (PCI) interfaces, local area network (LAN) interfaces, wide area network (WAN) interfaces, interfaces using serial protocols, interfaces using parallel protocols, asynchronous transfer mode interfaces, fiber distributed data interfaces (FDDI), interfaces based on transmission control protocol (TCP)/internet protocol (IP), interfaces based on wireless communications technology such as satellite technology, radio frequency technology, near field communication, etc.

The storage unit(s) 410 comprises non-transitory, computer-readable storage media, for example, fixed media drives such as hard drives, solid-state drives, etc., for storing an operating system, application programs, data volume files, etc. ; removable media drives for receiving removable media; etc. In an exemplary embodiment, the complete set of volumetric cross-sectional computer tomography (CT) or cone beam computed tomography (CBCT) images of a desired anatomical area, which are acquired in the Digital Imaging and Communications in Medicine (DICOM) format, are stored in the storage unit(s) 410. In another exemplary embodiment, the complete set of volumetric cross-sectional CT or CBCT images in the DICOM format is stored in the cloud storage 412 via the network 402. The common modules 409 comprise, for example, input/output (I/O) controllers, input devices, output devices, fixed media drives such as hard drives, removable media drives for receiving removable media, etc. The input devices comprise, for example, a keyboard such as an alphanumeric keyboard, a pointing device such as a computer mouse, a touch pad, a light pen, a digital pen, a microphone for providing voice input, a physical button, a touch sensitive display device, a track ball, a pointing stick, any device capable of sensing a tactile input, etc. The output devices output the results of operations performed by the scaffold designing engine 408. For example, the scaffold designing engine 408 renders a data volume, multiple complementary, cross-sectional image visualizations of the data volume and a three-dimensional (3D) image visualization of the data volume, a membrane, a wall surface, a mathematical digital representation of a two-dimensional (2D) outer surface of the implantable scaffold, etc., to users using the output devices. Computer applications and programs are used for operating the scaffold designing engine 408. The programs are loaded onto the fixed media drives and into the memory unit 407 via the removable media drives or downloaded from the network 402 or the cloud storage 412. In an exemplary embodiment, the computer applications and programs are loaded into the memory unit 407 directly via the network 402.

In an exemplary embodiment, the modules 408a to 408j of the scaffold designing engine 408 are computer-embeddable systems that design an outer shape of an implantable scaffold and plan fixation of the implantable scaffold. In the exemplary implementation illustrated in FIG. 4, the modules of the scaffold designing engine 408 comprise an image access module 408a, a data volume generator 408b, a defect contour generator 408c, a membrane generator 408d, a base generator 408e, a wall surface generator 408f, a 2D scaffold outer surface generator 408g, a fixation planning module 408h, and a database 408i. The database 408i is any storage area or medium that can be used for storing data, for example, volumetric data, voxelated data, etc., and files, for example, DICOM format files, data volume files, etc. The database 408i can be, for example, any of a structured query language (SQL) data store or a not only SQL (NoSQL) data store such as the Microsoft® SQL Server®, the Oracle® servers, the MySQL® database of MySQL AB Limited Company, the mongoDB® of MongoDB, Inc., the Neo4j graph database of Neo Technology Corporation, the Cassandra database of the Apache Software Foundation, the HBase® database of the Apache Software Foundation, etc. In an exemplary embodiment, the database 408i can also be a location on a file system. In another exemplary embodiment, the database 408i can be remotely accessed by the scaffold designing engine 408 via the network 402. In another exemplary embodiment, the database 408i is configured as a cloud-based database implemented in a cloud computing environment, where computing resources are delivered as a service over the network 402.

The image access module 408a accesses multiple cross-sectional medical diagnostic images fully covering a bone regeneration volume, previously acquired using appropriate computer tomography (CT) scanners, and stores the cross-sectional medical diagnostic images in the database 408i, and/or the storage unit(s) 410, and/or the cloud storage 412. The data volume generator 408b then generates a digital representation of the bone regeneration volume as a data volume comprising volumetric samplings of the cross-sectional medical diagnostic images and stores the data volume in the database 408i, and/or the storage unit(s) 410, and/or the cloud storage 412. The data volume generator 408b then renders multiple complementary, cross-sectional image visualizations of the data volume and a three-dimensional (3D) image visualization of the data volume as disclosed in the description of FIG. 1A. The defect contour generator 408c then identifies a digital representation of a defect contour separating a healthy bone surface from an outer surface of a bone defect subject to bone regeneration, using at least one of the rendered complementary, cross-sectional image visualizations of the data volume and the 3D image visualization of the data volume. In an exemplary embodiment, the defect contour generator 408c interactively identifies isolated seed points of the defect contour with a subsequent interpolation between the isolated seed points of the defect contour.

Furthermore, the membrane generator 408d renders a mathematical digital representation of the outer surface of the bone defect encircled by the defect contour, as a membrane. In an exemplary embodiment, the membrane generator 408d defines the membrane through a 3D interpolation of the defect contour into an interior of the defect contour using computer graphics and/or one or more mathematical algorithms. In another exemplary embodiment, the membrane generator 408d performs the 3D interpolation of the defect contour into the interior of the defect contour using a mathematical function built over local data of the data volume. In an exemplary embodiment, the 3D interpolation of the defect contour into an interior of the defect contour comprises employing interactively seeded anchor points in the membrane. That is, the membrane generator 408d performs the 3D interpolation in such a way that the membrane includes interactively seeded anchor points. In an exemplary embodiment, the membrane generator 408d configures the membrane to be adjacent to a bone surface only along and in vicinity of the defect contour, while being considerably separated from the healthy bone surface within an inner area of the membrane. The base generator 408e then identifies a spatial location and a normal vector of a first base plane configured to hold a first printed layer of the implantable scaffold. The base generator 408e further identifies an area of the first base plane enclosed within a projection of the defect contour onto the first base plane as a base facet of the implantable scaffold.

The wall surface generator 408f renders a first tubular surface extending from the defect contour onto the base facet. The wall surface generator 408f configures a wall surface as part of the first tubular surface confined between the base facet and the defect contour to air-tightly embrace the base facet at a first cross-section of the wall surface and air-tightly embrace the defect contour at a second cross-section of the wall surface. The 2D scaffold outer surface generator 408g then renders a mathematical digital representation of a 2D outer surface of the implantable scaffold by a contiguous joinder of a 2D segment of the base facet, a 2D segment of the membrane, and a 2D segment of the wall surface. The rendering of the mathematical digital representation of the 2D outer surface of the implantable scaffold constitutes the design of the outer shape of the implantable scaffold.

The fixation planning module 408h is configured to plan the fixation of the implantable scaffold. In an exemplary embodiment, the fixation planning module 408h identifies and provides a visualization of vulnerable anatomical structures in the vicinity of the implantable scaffold. The fixation planning module 408h selects an optimal location for a fixation member, for example, a fixation screw, of an optimal length to be disposed in an optimal orientation to ensure secure anchoring of the fixation member to a neighboring bone tissue, while preventing protrusion of the fixation member beyond a necessary anchoring volume into a dangerous proximity to the identified vulnerable anatomical structures. The fixation planning module 408h plans the fixation member safely beyond the dangerous proximity to the vulnerable anatomical structures. In this exemplary embodiment, the fixation planning module 408h renders an enhanced 2D outer surface of the implantable scaffold incorporating a pilot hole for the fixation member in the selected optimal location and the optimal orientation. In an exemplary embodiment, the fixation planning module 408h selects the optimal location, the optimal length, and the optimal orientation of the fixation member using one or more of: deterministic algorithms, artificial intelligence (AI) algorithms comprising deep learning algorithms, and interactive user input based on a visualization of an axis and a shape of the fixation member on the 3D image visualization and the cross-sectional image visualizations. In an exemplary embodiment, the fixation planning module 408h identifies a minimal proximity of the implant scaffold and/or the fixation member to the identified vulnerable anatomical structures, and generates an alert when the implant scaffold and/or the fixation member is in dangerous proximity to the identified vulnerable anatomical structures. The fixation planning module 408h allows and incorporates adjustments of the outer shape of the implantable scaffold and the location of the fixation member based on the alert. In an exemplary embodiment, the scaffold designing engine 408 further comprises a tracker 408j configured to track and log a complete history or a partial history of user actions, approvals, and selections for audit trails and further revisions of the rendered digital representations and image visualizations, in the storage unit(s) 410 or a persistent storage.

The processor(s) 405 in the computing system 403 retrieves instructions defined by the image access module 408a, the data volume generator 408b, the defect contour generator 408c, the membrane generator 408d, the base generator 408e, the wall surface generator 408f, the 2D scaffold outer surface generator 408g, the fixation planning module 408h, and the tracker 408j from the memory unit 407 for executing the respective functions disclosed above. The modules 408a to 408j in the computing system 403 are disclosed above as software executed by the processor(s) 405. In an exemplary embodiment, the modules 408a to 408j of the scaffold designing engine 408 are implemented completely in hardware. In another exemplary embodiment, the scaffold designing engine 408 is also implemented as a combination of hardware and software and one or more processors, for example, 405, that are used to implement the modules, for example, 408a to 408j, of the scaffold designing engine 408.

For purposes of illustration, the disclosure herein refers to the scaffold designing engine 408 being run locally on a single computing system 403; however the scope of the system 400 and the method disclosed herein is not limited to the scaffold designing engine 408 being run locally on a single computing system 403 via the operating system and the processor(s) 405, but extends to running the scaffold designing engine 408 remotely over the network 402 by employing a web browser, one or more remote servers, computers, mobile phones, and/or other electronic devices. In an exemplary embodiment, one or more portions of the computing system 403 are distributed across one or more computer systems (not shown) coupled to the network 402. In another exemplary embodiment, one or more modules, databases, processing elements, memory elements, storage elements, etc., of the system 400 disclosed herein are distributed across a cluster of computer systems (not shown), for example, computers, servers, virtual machines, containers, nodes, etc., coupled to the network 402, where the computer systems coherently communicate and coordinate with each other to share resources, distribute workload, and execute different portions of the logic to design an outer shape of an implantable scaffold.

The non-transitory, computer-readable storage medium disclosed herein stores computer program instructions executable by the processor(s) 405 for designing an outer shape of an implantable scaffold and planning fixation of the implantable scaffold. The computer program instructions implement the processes of various embodiments disclosed above and perform additional steps that may be required and contemplated for designing an outer shape of an implantable scaffold and planning fixation of the implantable scaffold. When the computer program instructions are executed by the processor(s) 405, the computer program instructions cause the processor(s) 405 to perform the steps of the method for designing an outer shape of an implantable scaffold and planning fixation of the implantable scaffold as disclosed in the descriptions of FIGS. 1A-1B. In an exemplary embodiment, a single piece of computer program code comprising computer program instructions performs one or more steps of the method disclosed in the descriptions of FIGS. 1A-1B. The processor(s) 405 retrieves these computer program instructions and executes them.

A module, or an engine, or a unit, as used herein, refers to any combination of hardware, software, and/or firmware. As an example, a module, or an engine, or a unit includes hardware such as a microcontroller, associated with a non-transitory, computer-readable storage medium to store computer program codes adapted to be executed by the microcontroller. Therefore, references to a module, or an engine, or a unit, in an exemplary embodiment, refer to the hardware that is specifically configured to recognize and/or execute the computer program codes to be held on a non-transitory, computer-readable storage medium. In an exemplary embodiment, the computer program codes comprising computer readable and executable instructions are implemented on any platform or in any programming language, for example, Windows®, Unix®, the iOS of Apple Inc., C, C++, Java®, Python®, etc. In another exemplary embodiment, other object-oriented, functional, scripting, and/or logical programming languages are also used. In an exemplary embodiment, the computer program codes or software programs are stored on or in one or more mediums as object code. In another exemplary embodiment, the term “module” or “engine” or “unit” refers to the combination of the microcontroller and the non-transitory, computer-readable storage medium. Often module or engine or unit boundaries that are illustrated as separate commonly vary and potentially overlap. For example, a module or an engine or a unit may share hardware, software, firmware, or a combination thereof, while potentially retaining some independent hardware, software, or firmware. In various embodiments, a module or an engine or a unit includes any suitable logic.

The system 400 comprising the scaffold designing engine 408 and the method disclosed herein provide an improvement in computer-aided design (CAD)/computer-aided manufacturing (CAM) dentistry. In the system 400 and the method disclosed herein, the design and the flow of interactions between the user devices 401a and 401b and the computing system 403 and between the modules 408a to 408j of the scaffold designing engine 408 are deliberate, designed, and directed. The cross-sectional medical diagnostic images that fully cover a bone regeneration volume, received by the scaffold designing engine 408, are configured by the scaffold designing engine 408 to steer the cross-sectional medical diagnostic images towards a finite set of outcomes. The scaffold designing engine 408 implements one or more specific computer programs to direct the cross-sectional medical diagnostic images towards a set of end results. The interactions designed by the scaffold designing engine 408 allow the scaffold designing engine 408 to generate the data volume, render the complementary, cross-sectional image visualizations of the data volume and the 3D image visualization of the data volume, identify a defect contour, render a membrane, identify a spatial location and a normal vector of a first base plane, identify a base facet, and render a first tubular surface and a corresponding wall surface; and from these digital representations, through the use of other, separate and autonomous computer programs, render a mathematical digital representation of a 2D outer surface, herein referred to as the “outer shape”, of the implantable scaffold. Furthermore, through the use of other, separate and autonomous computer programs, the scaffold designing engine 408 plans fixation of the implantable scaffold by identifying and providing a visualization of vulnerable anatomical structures in the vicinity of the implantable scaffold; selecting an optimal location for a fixation member of an optimal length to be disposed in an optimal orientation; rendering an enhanced 2D outer surface of the implantable scaffold incorporating a pilot hole for the fixation member in the selected optimal location and the optimal orientation; identifying a minimal proximity of the implantable scaffold and/or the fixation member to the identified vulnerable anatomical structures; and generating an alert when the implantable scaffold and/or the fixation member are in dangerous proximity to the identified vulnerable anatomical structures. To perform the above disclosed method steps requires multiple separate computer programs and subprograms, the execution of which cannot be performed by a person using a generic computer with a generic program. The focus of the system 400 and the method disclosed herein is on an improvement to CAD/CAM technology for designing an outer shape of an implantable scaffold and for planning fixation of the implantable scaffold, and not on economic or other tasks for which a generic computer is used in its ordinary capacity. Accordingly, the system 400 and the method disclosed herein are not directed to an abstract idea. Rather, the system 400 and the method disclosed herein are directed to a specific improvement to the way the scaffold designing engine 408 operates, embodied in, the method steps disclosed above.

The previous embodiment described above, illustrates a computer-assisted method of designing the outer shape of 3D printable construction of the implantable scaffold through interactive identification of the bone defect contour, the membrane surface adjacent to the bone defect and encircled with the defect contour and the base plane at a certain distance from and not intersecting with the defect contour; with further projection of such defined defect contour onto the base plane to define a base facet and then identifying a wall segment as a section of the first tubular surface extending from the defect contour up to the outer edge of the base faucet and contained between the defect contour and the base plane; and finally identifying the outer implantable scaffold shape as a watertight joinder of the base facet, wall surface and the membrane. Typical 3D representation of such designed surface is presented on FIGS. 2H-2J where 270 denotes the outer surface of the implantable scaffold, 271 denotes the membrane segment, 272 denotes the base facet, and 273 denoted the wall segment.

As clearly illustrates in FIGS. 2H and 2I, where the scaffold outer surface is presented in relation to the bone surface, such constructed implant allows for bone defect closure and bone augmentation in one direction - specifically the “extrusion direction” of the tubular wall segment.

However, in certain clinical situations it may be desirable to not only to close on the bone defect, but also to provide bone augmentation in two different directions. The following embodiment is focused on solving this specific clinical problem.

According to this embodiment, first the defect contour 220, the membrane 204, base plane, the base facet 272 and wall surface 273 are identified in full accordance with the workflow described in the first embodiment of the invention. Then, the contiguous joinder of a two-dimensional segment of the base facet 272, a two-dimensional segment of the membrane 204, and a two-dimensional segment of the wall surface 273 which within the scope of the first embodiment constituted the final outer shape of the implantable scaffold 270. Within the context of this embodiment the base facet 272 will be further identified as first base plane 272, while the outer shape of implantable scaffold 270 will be further identified as primary shell 2201, as shown in FIG. 2R. This primary shell 2201 is substantially identical to the scaffold's outer shape 270 constructed according to the first embodiment.

Then, user interactively identifies two additional objects: (1) the second base plane 2203 which is substantially perpendicular to the first base plane 272 and (2) the secondary extrusion vector 2202 belonging to the first base plane 272 and substantially perpendicular to the second base plane 2203 as shown in FIG. 2R. The second base plane 2203 acts as a secondary base plane and the normal vector 2202 of the secondary base plane acts as a secondary normal vector 2202.

Then the primary shell 2201 is projected onto the secondary base plane 2203 along the secondary normal vector 2202 thus identifying an area of the secondary base plane 2203 encompassing such a projection as secondary facet 2301, as shown in FIG. 2S.

Then, a second tubular surface is rendered through the extrusion or extension of the boundary of the secondary facet 2301 along the secondary normal vector 2202, as shown in FIG. 2S. A secondary wall surface 2302, as shown in FIG. 2S, is configured as part of the second tubular surface to air-tightly embrace the secondary facet 2301 at the first cross-section of the secondary wall surface 2302, and air-tightly embrace the primary shell 2201 at the second cross-section of the secondary wall surface 2302.

Then the equator contour 2401 of the primary shell 2201 is identified as the tangent curve resulting from intersection of the primary shell 2201 with the second wall surface 2302 as shown on FIG. 2T.

Then two half-shells of the primary shell 2201 are identified as two separate non-overlapping segments of the primary shell 2201 air-tightly joining each other over the equator contour - half-shell A 2402 and half-shell B 2403 as shown on FIG. 2T. Then two test constructions are created for further consideration. Construction A is constructed as an airtight joinder of a secondary facet 2301, second wall surface 2302 and Half-shell A 2402. Construction B is constructed as an airtight joinder of a secondary facet 2301, second wall surface 2302 and the Half-shell B 2403. One of the constructions, for example, Construction A will completely embrace and contain the other construction, for example, Construction B in this case; while the other construction, for example, Construction B in this case, would be completely contained within that first Construction A. Respectively, the Half-shell A 2402 may be referred to as outer half-shell, while half-shell B 2403 may be referred to as inner half-shell.

Then a digital representation of a two-dimensional outer surface of the implantable scaffold is rendered by a contiguous joinder of a two-dimensional segment of the secondary facet 2301, a two-dimensional segment of the outer half-shell, and a two-dimensional segment of the second wall surface 2302.

The method and the system disclosed herein are directed towards seamless designing of printable, implantable scaffolds compromising the universality of conventional CAD/CAM applications for flexible engineering mechanical parts and shapes of any type. The method and the system disclosed herein allow a medical practitioner, who may generally lack knowledge in engineering methods, to design the outer shape of the implantable scaffold and plan the fixation of the implantable scaffold using familiar and meaningful tools and concepts similar to those used for daily medical imaging routines, rather than struggling through engineering abstractions and concepts. The method and the system disclosed herein also preclude tedious manual engineering of elementary surfaces comprising the outer shape of the implantable scaffold and then manually joining the elementary surfaces in a separate step, by outlining simpler shapes of lesser dimensions and complexity, for example, the defect contour, the extrusion vector, scaffold thickness, etc., while the actual elementary surfaces and their joinder are built automatically. Moreover, the method and the system disclosed herein perform automatic identification of the proximity of the implantable scaffold and the fixation member to vulnerable anatomical structures and issues an alert or a warning message when the implantable scaffold and the fixation member are in dangerous proximity to the vulnerable anatomical structures. Furthermore, the method and the system disclosed herein allow adjustments of the shape of the implantable scaffold and the location of the fixation member (a) at any step of design; (b) via a few intuitive controls rather than a tedious redesign of elementary parts with a further reassembly in one joinder; and (c) with immediate visualization of the adjusted surface rather than visualization of only one elementary part thereof. The method and the system disclosed herein do not create a stereolithography (STL) model of the bone, but rather visualizing the bone as a cloud by using a ray tracing volumetric representation of the bone or via any other method customary to the common applications for medical images visualization and analysis. The implantable scaffold produced by the method and the system disclosed herein has a high success rate of bone regeneration with reduced cost.

It is apparent in different embodiments that the various methods, algorithms, and computer-readable programs disclosed herein are implemented on non-transitory, computer-readable storage media appropriately programmed for computing devices. The non-transitory, computer-readable storage media participate in providing data, for example, instructions that are read by a computer, a processor, or a similar device. In different embodiments, the “non-transitory, computer-readable storage media” also refer to a single medium or multiple media, for example, a centralized database, a distributed database, and/or associated caches and servers that store one or more sets of instructions that are read by a computer, a processor, or a similar device. The “non-transitory, computer-readable storage media” also refer to any medium capable of storing or encoding a set of instructions for execution by a computer, a processor, or a similar device and that causes a computer, a processor, or a similar device to perform any one or more of the steps of the method disclosed herein. In an exemplary embodiment, the computer programs that implement the methods and algorithms disclosed herein are stored and transmitted using a variety of media, for example, the computer-readable media in various manners. In an exemplary embodiment, hard-wired circuitry or custom hardware is used in place of, or in combination with, software instructions for implementing the processes of various embodiments. Therefore, the embodiments are not limited to any specific combination of hardware and software. Various aspects of the embodiments disclosed herein are implemented in a non-programmed environment comprising documents created, for example, in a hypertext markup language (HTML), an extensible markup language (XML), or other format that render aspects of a graphical user interface (GUI) or perform other functions, when viewed in a visual area or a window of a browser program. Various aspects of the embodiments disclosed herein are implemented as programmed elements, or non-programmed elements, or any suitable combination thereof.

Where databases are described such as the database 408i, it will be understood by one of ordinary skill in the art that (i) alternative database structures to those described may be employed, and (ii) other memory structures besides databases may be employed. Any illustrations or descriptions of any sample databases disclosed herein are illustrative arrangements for stored representations of information. In an exemplary embodiment, any number of other arrangements are employed besides those suggested by tables illustrated in the drawings or elsewhere. In another exemplary embodiment, despite any depiction of the databases as tables, other formats including relational databases, object-based models, and/or distributed databases are used to store and manipulate the data types disclosed herein. In an exemplary embodiment, object methods or behaviors of a database are used to implement various processes such as those disclosed herein. In another exemplary embodiment, the databases are, in a known manner, stored locally or remotely from a device that accesses data in such a database. In embodiments where there are multiple databases, the databases are integrated to communicate with each other for enabling simultaneous updates of data linked across the databases, when there are any updates to the data in one of the databases.

The embodiments disclosed herein are configured to operate in a network environment comprising one or more computers that are in communication with one or more devices via a network. In an exemplary embodiment, the computers communicate with the devices directly or indirectly, via a wired medium or a wireless medium such as the Internet, satellite internet, a local area network (LAN), a wide area network (WAN) or the Ethernet, or via any appropriate communications mediums or combination of communications mediums. Each of the devices comprises processors that are adapted to communicate with the computers. In an exemplary embodiment, each of the computers is equipped with a network communication device, for example, a network interface card, a network interface microchip, a modem, or other network connection device suitable for connecting to a network. Each of the computers and the devices executes an operating system. While the operating system may differ depending on the type of computer, the operating system provides the appropriate communications protocols to establish communication links with the network. Any number and type of machines may be in communication with the computers.

The embodiments disclosed herein are not limited to a particular computer system platform, processor, operating system, or network. One or more of the embodiments disclosed herein are distributed among one or more computer systems, for example, servers configured to provide one or more services to one or more client computers, or to perform a complete task in a distributed system. For example, one or more of the embodiments disclosed herein are performed on a client-server system that comprises components distributed among one or more server systems that perform multiple functions according to various embodiments. These components comprise, for example, executable, intermediate, or interpreted code, which communicate over a network using a communication protocol. The embodiments disclosed herein are not limited to be executable on any particular system or group of systems, and are not limited to any particular distributed architecture, network, or communication protocol.

The foregoing examples and illustrative implementations of various embodiments have been provided merely for explanation and are in no way to be construed as limiting the embodiments disclosed herein. While the embodiments have been described with reference to various illustrative implementations, drawings, and techniques, it is understood that the words, which have been used herein, are words of description and illustration, rather than words of limitation. Furthermore, although the embodiments have been described herein with reference to particular means, materials, techniques, and implementations, the embodiments herein are not intended to be limited to the particulars disclosed herein; rather, the embodiments extend to all functionally equivalent structures, methods and uses, such as are within the scope of the appended claims. It will be understood by those skilled in the art, having the benefit of the teachings of this specification, that the embodiments disclosed herein are capable of modifications and other embodiments may be effected and changes may be made thereto, without departing from the scope and spirit of the embodiments disclosed herein.

We claim:

1. A method employing a scaffold designing engine configured to implement computer program instructions executable by at least one processor for designing an outer shape of an implantable scaffold, the method comprising:
acquiring and storing a plurality of cross-sectional medical diagnostic images fully covering a bone regeneration volume in a storage unit;
generating a digital representation of the bone regeneration volume as a data volume comprising volumetric samplings of the plurality of cross-sectional medical diagnostic images and storing the data volume in the storage unit;
rendering a plurality of complementary, cross-sectional image visualizations of the data volume and a three-dimensional image visualization of the data volume, wherein the plurality of complementary, cross-sectional image visualizations of the data volume comprise planar cross-sectional sampling of the data volume, and wherein the three-dimensional image visualization of the data volume comprises one of: visualizations of three-dimensional data iso-surfaces built over data of the data volume, visualizations of two-dimensional projections of ray tracing models built over the data of the data volume, and any combination thereof;
identifying a digital representation of a defect contour separating a healthy bone surface from an outer surface of a bone defect subject to the bone regeneration, using at least one of the plurality of complementary, cross-sectional image visualizations of the data volume and the three-dimensional image visualization of the data volume;
rendering a digital representation of the outer surface of the bone defect encircled by the defect contour, as a membrane;
identifying a spatial location and a normal vector of a first base plane configured to hold a first printed layer of the implantable scaffold, and further identifying an area of the first base plane enclosed within a projection of the defect contour onto the first base plane as a base facet of the implantable scaffold;
rendering a first tubular surface extending from the defect contour onto the base facet, and configuring a wall surface as part of the first tubular surface confined between the base facet and the defect contour to air-tightly embrace the base facet at a first cross-section of the wall surface and air-tightly embrace the defect contour at a second cross-section of the wall surface; and
rendering a digital representation of a two-dimensional outer surface of the implantable scaffold by a contiguous joinder of a two-dimensional segment of the base facet, a two-dimensional segment of the membrane, and a two-dimensional segment of the wall surface.

2. The method of claim 1, wherein the first tubular surface is a classical mathematical cylinder defined by the defect contour as a generatrix and an extrusion vector that is substantially parallel to the normal vector to the first base plane, and wherein a border of the base facet is defined as a projection of the defect contour onto the first base plane along the extrusion vector.

3. The method of claim 1, wherein the scaffold designing engine is configured to execute one or more of mathematical algorithms and artificial intelligence algorithms comprising deep learning methods for implementing one or more steps of the method for designing the outer shape of the implantable scaffold.

4. The method of claim 1, further comprising defining the membrane through a three-dimensional interpolation of the defect contour into an interior of the defect contour using computer graphics.

5. The method of claim 1, further comprising defining the membrane through a three-dimensional interpolation of the defect contour into an interior of the defect contour by employing interactively seeded anchor points in the membrane.

6. The method of claim 1, wherein user interactions are used for implementing one or more steps of the method for designing the outer shape of the implantable scaffold, wherein the user interactions are performed over two-dimensional and three-dimensional computer visualizations of the digital representation of the bone regeneration volume, the plurality of complementary, cross-sectional image visualizations of the data volume and the three-dimensional image visualization of the data volume, the digital representation of the outer surface of the bone defect encircled by the defect contour, the first tubular surface, and the digital representation of the two-dimensional outer surface of the implantable scaffold.

7. The method of claim 6, further comprising employing the plurality of complementary, cross-sectional image visualizations of the data volume along three mutually perpendicular cross-sectional planes.

8. The method of claim 7, further comprising interactively adjusting orientation of the three mutually perpendicular cross-sectional planes.

9. The method of claim 1, further comprising employing visualizations of one of:
the three-dimensional data iso-surfaces within the data volume;
iso-surfaces of a combination of the data within the data volume and spatial derivatives of the data; and
iso-surfaces of complex mathematical functions over the data volume in addition to the combination of the data within the data volume and the spatial derivatives of the data.

10. The method of claim 1, further comprising employing a visualization of a digital simulation of optical rays penetrating through an optically nonuniform volume, wherein local optical characteristics are mathematically related to local values within the data volume.

11. The method of claim 1, further comprising interactively identifying isolated seed points of the defect contour with a subsequent interpolation between the isolated seed points of the defect contour.

12. The method of claim 1, wherein the membrane is configured to be adjacent to a bone surface only along and in a vicinity of the defect contour, while being considerably separated from the healthy bone surface within an inner area of the membrane.

13. The method of claim 1, further comprising identifying vulnerable anatomical structures using one of: one or more user interactions, artificial intelligence algorithms comprising deep learning algorithms, and any combination thereof.

14. The method of claim 13, further comprising:
identifying a minimal proximity of the implantable scaffold to the identified vulnerable anatomical structures; and
generating an alert when the implantable scaffold is in dangerous proximity to the identified vulnerable anatomical structures.

15. The method of claim 1, further comprising:
selecting an optimal location for a fixation member of an optimal length to be disposed in an optimal orientation to ensure secure anchoring of the fixation member to a neighboring bone tissue, while preventing protrusion of the fixation member beyond a necessary anchoring volume into a dangerous proximity to vulnerable anatomical structures; and
rendering an enhanced two-dimensional outer surface of the implantable scaffold incorporating a pilot hole for the fixation member in the selected optimal location and the optimal orientation.

16. The method of claim 15, further comprising:
identifying a minimal proximity of one or more of the implantable scaffold and the fixation member to the vulnerable anatomical structures; and
generating an alert when the one or more of the implantable scaffold and the fixation member are in dangerous proximity to the vulnerable anatomical structures.

17. The method of claim 15, wherein the optimal location, the optimal length, and the optimal orientation of the fixation member are selected using one or more of: deterministic algorithms, artificial intelligence algorithms comprising deep learning algorithms, and interactive user input based on a visualization of an axis and a shape of the fixation member on the three-dimensional image visualization and the plurality of complementary, cross-sectional image visualizations of the data volume.

18. The method of claim 1 further comprising:
identifying the outer surface resulting from the contiguous joinder of the two-dimensional segment of the base facet, the two-dimensional segment of the membrane, and the two-dimensional segment of the wall surface as a primary shell;
identifying a spatial location and a normal vector of a second base plane, wherein the second base plane is substantially perpendicular to the first base plane and acts as a secondary base plane and the normal vector of the secondary base plane acts as a secondary normal vector;
projecting the primary shell on the second base plane along the secondary normal vector and identifying an area of the second base plane encompassing such a projection as a secondary facet;
rendering a second tubular surface extending from a boundary of the secondary facet along the secondary normal vector, and configuring a secondary wall surface as part of the second tubular surface to air-tightly embrace the secondary facet at the first cross-section of the secondary wall surface and air-tightly embrace the primary shell at the second cross-section of the secondary wall surface;
identifying an equator contour of the primary shell as a tangent curve resulting from intersection of the primary shell with the second tubular surface;
identifying two half-shells of the primary shell as two separate non-overlapping segments of the primary shell air-tightly joining each other over the equator contour;
identifying one of the two half-shells that is fully contained within an inner volume confined by the airtight joinder of the secondary facet, secondary wall surface and other half-shell, as inner half-shell while the other half-shell as outer half-shell; and
rendering a digital representation of a two-dimensional outer surface of the implantable scaffold by a contiguous joinder of a two-dimensional segment of the secondary facet, a two-dimensional segment of the outer half-shell, and a two-dimensional segment of the secondary wall surface.

19. A method employing a scaffold designing engine configured to implement computer program instructions executable by at least one processor for designing and planning fixation of an implantable scaffold, the method comprising:
designing an outer shape of the implantable scaffold, comprising:
acquiring and storing a plurality of cross-sectional medical diagnostic images fully covering a bone regeneration volume in a storage unit;
generating a digital representation of the bone regeneration volume as a data volume comprising volumetric samplings of the plurality of cross-sectional medical diagnostic images and storing the data volume in the storage unit;
rendering a plurality of complementary, cross-sectional image visualizations of the data volume and a three-dimensional image visualization of the data volume, wherein the plurality of complementary, cross-sectional image visualizations of the data volume comprise planar cross-sectional sampling of the data volume, and wherein the three dimensional image visualizations comprises one of: visualizations of three-dimensional data iso-surfaces built over data of the data volume, visualizations of two-dimensional projections of ray tracing models built over the data of the data volume, and any combination thereof;
identifying a digital representation of a defect contour separating a healthy bone surface from an outer surface of a bone defect subject to the bone regeneration, using at least one of the plurality of complementary, cross-sectional image visualizations of the data volume and the three-dimensional image visualization of the data volume;
rendering a digital representation of the outer surface of the bone defect encircled by the defect contour, as a membrane;
identifying a spatial location and a normal vector of a first base plane configured to hold a first printed layer of the implantable scaffold, and further identifying an area of the first base plane enclosed within a projection of the defect contour onto the first base plane as a base facet of the implantable scaffold;
rendering a first tubular surface extending from the defect contour onto the base facet, and configuring a wall surface as part of the first tubular surface confined between the base facet and the defect contour to air-tightly embrace the base facet at a first cross-section of the wall surface and air-tightly embrace the defect contour at a second cross-section of the wall surface; and
rendering a digital representation of a two-dimensional outer surface of the implantable scaffold by a contiguous joinder of a two-dimensional segment of the base facet, a two-dimensional segment of the membrane, and a two-dimensional segment of the wall surface; and
planning the fixation of the implantable scaffold, comprising:
identifying and providing a visualization of vulnerable anatomical structures in a vicinity of the implantable scaffold;
selecting an optimal location for a fixation member of an optimal length to be disposed in an optimal orientation to ensure secure anchoring of the fixation member to a neighboring bone tissue, while preventing protrusion of the fixation member beyond a necessary anchoring volume into a dangerous proximity to the identified vulnerable anatomical structures;
rendering an enhanced two-dimensional outer surface of the implantable scaffold incorporating a pilot hole for the fixation member in the selected optimal location and the optimal orientation;
identifying a minimal proximity of one or more of the implantable scaffold and the fixation member to the identified vulnerable anatomical structures;
generating an alert when the one or more of the implantable scaffold and the fixation member are in dangerous proximity to the identified vulnerable anatomical structures; and
allowing and incorporating adjustments of the outer shape of the implantable scaffold and the location of the fixation member based on the alert.

20. A system for designing an outer shape of an implantable scaffold, the system comprising:
at least one processor;
a memory unit operably and communicatively coupled to the at least one processor and configured to store computer program instructions executable by the at least one processor; and
a scaffold designing engine implementing the computer program instructions, which when executed by the at least one processor, cause the at least one processor to:
acquire and store a plurality of cross-sectional medical diagnostic images fully covering a bone regeneration volume in a storage unit;
generate a digital representation of the bone regeneration volume as a data volume comprising volumetric samplings of the plurality of cross-sectional medical diagnostic images and store the data volume in the storage unit;
render a plurality of complementary, cross-sectional image visualizations of the data volume and a three-dimensional image visualization of the data volume, wherein the plurality of complementary, cross-sectional image visualizations of the data volume comprise planar cross-sectional sampling of the data volume, and wherein the three-dimensional image visualization of the data volume comprises one of: visualizations of three-dimensional data iso-surfaces built over data of the data volume, visualizations of two-dimensional projections of ray tracing models built over the data of the data volume, and any combination thereof;
identify a digital representation of a defect contour separating a healthy bone surface from an outer surface of a bone defect subject to bone regeneration, using at least one of the plurality of complementary, cross-sectional image visualizations of the data volume and the three-dimensional image visualization of the data volume;
render a digital representation of the outer surface of the bone defect encircled by the defect contour, as a membrane;
identify a spatial location and a normal vector of a first base plane configured to hold a first printed layer of the implantable scaffold, and further identifying an area of the first base plane enclosed within a projection of the defect contour onto the first base plane as a base facet of the implantable scaffold;
render a tubular surface extending from the defect contour onto the base facet, and configure a wall surface as part of the tubular surface confined between the base facet and the defect contour to air-tightly embrace the base facet at a first cross-section of the wall surface and air-tightly embrace the defect contour at a second cross-section of the wall surface; and
render a digital representation of a two-dimensional outer surface of the implantable scaffold by a contiguous joinder of a two-dimensional segment of the base facet, a two-dimensional segment of the membrane, and a two-dimensional segment of the wall surface.

21. The system of claim 20, configured to operate and be implemented in a cloud computing environment comprising a cloud storage for storing and accessing the plurality of cross-sectional medical diagnostic images.

22. The system of claim 20, wherein one or more of the computer program instructions implemented by the scaffold designing engine, which when executed by the at least one processor, cause the at least one processor to track and log one of a complete history and a partial history of user actions, approvals, and selections for audit trails and further revisions of the rendered digital representations and image visualizations, in the storage unit.
